(19) United States
(12) Patent Application Publication
(10) Pub. No.: US 2021/0386709 A1
(43) Pub. Date: Dec. 16, 2021

(54) COMPOSITIONS AND METHODS FOR SUPPRESSING MSUT2

(71) Applicants: United States Government As Represented By The Department of Veterans Affairs, Washington, DC (US); UNIVERSITY OF WASHINGTON, Seattle, WA (US)

(72) Inventors: JEREMY D. BAKER, Seattle, WA (US); Rikki L. Uhrich, Seattle, WA (US); Timothy J. Stovas, Seattle, WA (US); Aleen D. Saxton, Seattle, WA (US); Brian C. Kraemer, Seattle, WA (US)

(21) Appl. No.: 17/319,500

(22) Filed: May 13, 2021

Related U.S. Application Data
(60) Provisional application No. 63/024,117, filed on May 13, 2020.

(51) Int. Cl. A61K 31/41; A61P 25/28; A61K 31/381; A61K 31/4709; A61K 31/4985; A61K 31/4965; A61K 31/40; A61K 31/497; A61K 31/4748; A61K 31/192; A61K 31/55; A61K 31/27; A61K 31/445; C12Q 1/6804

(52) U.S. Cl. CPC A61K 31/41; A61P 25/28; A61K 31/381; A61K 31/4709; A61K 31/4985; A61K 31/4965; C12Q 1/6804; A61K 31/497; A61K 31/4748; A61K 31/192; A61K 31/55; A61K 31/27; A61K 31/445; A61K 31/40

(57) ABSTRACT

Described herein are compositions and methods for treating Alzheimer's disease, a tauopathy disorder or dementia. The compositions include mammalian suppressor of taupathy 2 (MSUT2) inhibitors. The methods include steps for identifying candidate compositions capable of inhibiting RNA binding proteins to poly(A) RNA and detecting RNA polyadenylation of poly(A) RNA. The methods include reducing accumulation of phosphorylated and aggregated human tau.

### CROSS REFERENCE TO RELATED APPLICATIONS

This application claims the benefit of U.S. Provisional Application No. 63/024,117, filed May 13, 2020. The content of this earlier filed application is hereby incorporated by reference herein in its entirety.

### STATEMENT REGARDING FEDERALLY FUNDED RESEARCH

This invention was made with government support under grant number I01BX002619 awarded by the Department of Veterans Affairs and under grant numbers R56AG057642 and RF1AG055474 awarded by National Institutes of Health. The government has certain rights in the invention.

### INCORPORATION OF THE SEQUENCE LISTING

The present application contains a sequence listing that is submitted via EFS-Web concurrent with the filing of this application, containing the file name “37759 0303U2 SL.txt” which is 4,096 bytes in size, created on May 12, 2021, and is herein incorporated by reference in its entirety.

### BACKGROUND

The brain protein tau, a natively unstructured protein encoded by the MAPT gene, performs an important physiological role in neurons by binding to and modulating neuronal microtubule stability (Brunden, K. R. et al., Nature Reviews Drug Discovery 2009, 8 (10), 783-793; Baas, P. W. et al., Trends Cell Biol 2019, 29 (6), 452-461; Gustke, N. et al., Biochemistry 1994, 33 (32), 9511-9522; and Binder, L. I., et al., J Cell Biol 1985, 101 (4), 1371-8). This activity helps to support the extensive processes neurons extend to conduct neuronal chemical and electrical signaling through axons (Inner, A. et al., Neuron 2018, 99 (1), 13-27; and Frere, S., et al. Neuron 2018, 97 (1), 32-58). Under neuronal stress or in disease states, tau is often hyper-phosphorylated or altered by other post-translational modifications (PTMs) resulting in a propensity to self-associate and produce detergent insoluble protein aggregates including paired helical filaments and neurofibrillary tangles (NFTs) (Fontaine, S. N. et al., Cell Mol Life Sci 2015, 72 (10), 1863-79; and Sabbagh, J. J. et al., Frontiers in Neuroscience 2016, 10 (3)). Neurons exhibit complex patterns of tau expression with multiple splice isoforms and a myriad of PTMs controlling tau function (Goedert, M. et al., Neuron 1989, 3 (4), 519-526; Wang, J.-Z., et al. Nature Medicine 1996, 2 (8), 871-875; and Wang, Y. et al., Nature Reviews Neuroscience 2016, 17 (1), 22-35). Tau deposits may take many pathological forms depending on the associated disorder. Tauopathies, or disorders with primary insoluble tau deposits as hallmarks, include Alzheimer's disease, Pick disease, progressive supranuclear palsy, corticobasal degeneration, chronic traumatic encephalopathy, and globular glial tauopathy (Strang, K. H. et al., Laboratory Investigation 2019, 99 (7), 912-928; and Iqbal, K., et al. Tau and neurodegenerative disease: the story so far. Nature Reviews Neurology 2016, 12 (1), 15-27). Distinct morphology of tau inclusions, molecular tau species, and brain regional distribution of tau containing lesions differentiate different tauopathy disorder subtypes (Lee, V. M. Y., et al., Annual Review of Neuroscience 2001, 24 (1), 1121-1159)). For example, Pick's disease pathology is primarily composed of spherical silver-positive aggregates of tau composed of the 3R tau isoform (Pick bodies) (Falcon, B., et al., Nature 2018, 561 (7721), 137-140), while progressive supranuclear palsy consists of neurofibrillary tangles as well as neuropil threads composed of the 4R tau isoform (Espinoza, M., et al. J Alzheimers Dis 2008, 14 (1), 1-16; and Buée, L., et al., Brain Pathology 1999, 9 (4), 681-693). Alzheimer's disease, the primary cause of dementia worldwide is a complex syndrome and hallmarks include NFTs, neuropil threads as well as tau-containing neuritic plaques (Iqbal, K., et al. Nature Reviews Neurology 2016, 12 (1), 15-27; and Henstridge, C. M., et al., Nature Reviews Neuroscience 2019, 20 (2), 94-108)). There are no disease-modifying therapeutics for ameliorating pathological tau; new mechanistic targets and therapeutic strategies for these disorders are desperately needed (Brunden, K. R., et al., Nature Reviews Drug Discovery 2009, 8 (10), 783-793; Congdon, E. E. et al., Nature Reviews Neurology 2018, 14 (7), 399-415; Cummings, J. L et. al., Alzheimers Res Ther 2014, 6 (4), 37-37; and Rojas, J. C., et al., Nature Reviews Neurology 2016, 12 (2), 74-76)).

### SUMMARY

Disclosed herein are methods of treating Alzheimer's disease, a tauopathy disorder or dementia, the methods comprising: administering to a subject with Alzheimer's disease, a tauopathy disorder or dementia a therapeutically effective amount of one or more of the mammalian suppressor of tauopathy 2 (MSUT2) inhibitors listed in Table 1, wherein the therapeutically effective amount reduces accumulation of phosphorylated and aggregated human tau.

Disclosed herein are methods of inhibiting expression of a MSUT2 polynucleotide in a subject, the methods comprising administering to a subject with Alzheimer's disease, a tauopathy disorder or dementia a therapeutically effective amount of one or more of the mammalian suppressor of tauopathy 2 (MSUT2) inhibitors listed in Table 1.

Disclosed herein are methods of inhibiting expression of a MSUT2 polynucleotide, the methods comprising contacting a cell with one or more of the mammalian suppressor of tauopathy 2 (MSUT2) inhibitors listed in Table 1, wherein the one or more of the mammalian suppressor of tauopathy 2 (MSUT2) inhibitors reduces accumulation of phosphorylated and aggregated tau.

Disclosed herein are methods of reducing phosphorylated and aggregated human tau protein in a subject, the methods comprising administering to a subject with Alzheimer's disease, a tauopathy disorder or dementia a therapeutically effective amount one or more of the mammalian suppressor of tauopathy 2 (MSUT2) inhibitors listed in Table 1.

Disclosed herein are methods of suppressing expression of a MSUT2 polynucleotide in a subject, the methods comprising administering to a subject with Alzheimer's disease, a tauopathy disorder or dementia a therapeutically effective amount of one or more of the mammalian suppressor of tauopathy 2 (MSUT2) inhibitors listed in Table 1.

Disclosed herein are methods of suppressing expression of a MSUT2 polynucleotide, the methods comprising contacting a cell with one or more of the mammalian suppressor of tauopathy 2 (MSUT2) inhibitors listed in Table 1, wherein the one or more of the mammalian suppressor of tauopathy 2 (MSUT2) inhibitors reduce accumulation of phosphorylated and aggregated tau.

Disclosed herein are methods of potentiating a neuroinflammatory response to a pathological tau protein in a subject, the methods comprising administering to a subject with Alzheimer's disease, a tauopathy disorder or dementia a therapeutically effective amount of one or more of the mammalian suppressor of tauopathy 2 (MSUT2) inhibitors listed in Table 1.

Disclosed herein are methods of potentiating a neuroinflammatory response to a pathological tau protein, the methods comprising contacting a cell with one or more of the mammalian suppressor of tauopathy 2 (MSUT2) inhibitors listed in Table 1, wherein the one or more of the mammalian suppressor of tauopathy 2 (MSUT2) inhibitors reduce accumulation of phosphorylated and aggregated tau.

Disclosed herein are methods of decreasing astrocytosis or microgliosis in a subject, the methods comprising administering to a subject with Alzheimer's disease, a tauopathy disorder or dementia a therapeutically effective amount of one or more of the mammalian suppressor of tauopathy 2 (MSUT2) inhibitors listed in Table 1.

Disclosed herein are methods of decreasing astrocytosis or microgliosis, the methods comprising contacting a cell with one or more of the mammalian suppressor of tauopathy 2 (MSUT2) inhibitors listed in Table 1, wherein the one or more of the mammalian suppressor of tauopathy 2 (MSUT2) inhibitors reduces accumulation of phosphorylated and aggregated tau.

Disclosed herein are methods of reducing neuroinflammation in a subject, the methods comprising administering to a subject with Alzheimer's disease, a tauopathy disorder or dementia a therapeutically effective amount of one or more of the mammalian suppressor of tauopathy 2 (MSUT2) inhibitors listed in Table 1.

Disclosed herein are methods of reducing neuroinflammation, the methods comprising contacting a cell with one or more of the mammalian suppressor of tauopathy 2 (MSUT2) inhibitors listed in Table 1, wherein the one or more of the mammalian suppressor of tauopathy 2 (MSUT2) inhibitors reduce accumulation of phosphorylated and aggregated tau.

Disclosed herein are methods of screening for compounds capable of inhibiting MSUT2 binding to poly(A) RNA, the methods comprising: (a) contacting at least one candidate compound, poly(A) RNA and PABPN1 under conditions in which PABPN1 is capable of stimulating RNA polyadenylation in the absence of the candidate compound; (b) determining whether the candidate compound inhibits MSUT2 binding to poly(A) RNA; and (c) selecting the candidate compound which inhibits MSUT2 binding to poly(A) RNA.

Disclosed herein are methods for screening compounds for pharmacological intervention in tauopathy disorders, the methods comprising: (a) providing an assay for MSUT2 to bind to poly(A) RNA and its modulation of RNA polyadenylation; (b) providing a purified or non-purified compound or purified or non-purified mixture of compounds; (c) screening the purified or non-purified compound or purified or non-purified mixture of compounds in an environment that allow for inhibition of MSUT2 binding to poly(A) RNA by the purified or non-purified compound or purified or non-purified mixture of compounds in the assay; and (d) identifying one or more compounds that inhibit MSUT2 binding to poly(A) RNA.

Disclosed herein are methods of detecting RNA polyadenylation of poly(A) RNA, the methods comprising: a) providing at least one candidate compound; b) providing at least one poly(A) RNA molecule bound to at least one donor component, wherein the at least one donor component emits a signal after it is irradiated by a light source; c) providing at least one MSUT2 polypeptide bound to at least one acceptor component, wherein the at least one acceptor component is able to receive a signal from the at least one donor component and emit a signal in the form of an electromagnetic radiation; d) bringing the at least one candidate compound, the at least one poly(A) RNA molecule bound to at least one donor component; and the at least one MSUT2 polypeptide bound to at least one acceptor component in contact with each other; e) irradiating the mixture of step d) with a light source; and f) measuring the electromagnetic radiation emitted by the mixture.

Disclosed herein are methods for identifying a candidate composition capable of inhibiting a RNA binding protein (RBP) binding to poly(A) RNA, the methods comprising: a) contacting a fluorescent probe molecule bound to the poly(A)RNA molecule (FAM-RNA), the RBP, and the candidate composition in a sample under conditions in which the RBP is capable of binding to the FAM-RNA molecule and forming a macromolecular complex, wherein the macromolecular complex comprises FAM-RNA:RBP; b) exciting fluorescence in the sample with linearly polarized light from a pulsed excitation source; c) detecting a fluorescent emission from the excited sample; and d) measuring anisotropy of the emitted fluorescence, wherein the reduction of emitted polarized fluorescence identifies a candidate composition capable of inhibiting a RNA binding protein (RBP) binding to poly(A) RNA.

Other features and advantages of the present compositions and methods are illustrated in the description below, the drawings, and the claims.

### BRIEF DESCRIPTION OF THE DRAWINGS

FIGS. 1A-C show the underlying Alpha Assay methodology for identifying inhibitory compounds. FIG. 1A shows a schematic of Alpha Assay. Excitation at wavelength 680 nm results in the conversion of ambient oxygen to excited singlet oxygen by the donor bead (coated with streptavidin/biotinylated poly(A) RNA). If the donor bead is in close proximity (˜200 nm) to the acceptor bead (coated with glutathione/GST-conjugated MSUT2), the singlet oxygen excites the acceptor bead resulting in light emission at 615 nm. Inhibiting this reaction with compounds stops beads coming near one another and results in lower or no emission. FIG. 1B shows that Z′factor for the screen was calculated at 0.85, σ=standard deviation. FIG. 1C shows a schematic of hit selection methodology. A mixture of donor beads, biotinylated poly(A) RNA and GST-MSUT2 was plated in 384 well format at 12 uL/well volume. 50 nL of compounds were added via pin tool and incubated at room temperature for 30 minutes away from light, at which point 3 uL of acceptor beads were added to wells. Plates were again incubated at room temperature away from light for 60 minutes. Plates were read with Perkin Elmer Envision using standard Alpha Assay protocol and data was analyzed. Hits were then validated for dose response, in an orthogonal fluorescence polarization assay, for specificity, and finally for cellular toxicity.

FIGS. 2A-B show the Spectrum Collection Alpha Screen. FIG. 2A shows the 2000 compound Spectrum collection that was screened using Alpha Screen. Hit window was considered the compounds more than 7 standard deviations from the mean. FIG. 2B shows the structures of the 20 small molecules identified from the Spectrum Collection.

FIGS. 3A-B show the Alpha Assay dose response validation for hits, where Y axis is % inhibition and X axis is concentration of the indicated compound as Molarity. FIG. 3A shows the results for 2,3-dichloro-5,8-dihydroxynapthoquinone, 3,4-didesmethyl-5-deshydroxy-3′ethoxyscleroin, aurin tricarboxylic acid, chloranil, ethacridine lactate, irigenol, mitoxantrone hydrochloride, nisoldipine, pyrogallin, and tannic acid. FIG. 3B shows the results for 4,4′-diisothiocyanostilbene-2′2′-sulfonic acid sodium salt, alizarin, doxorubicin, ebselen, koparin, methylene blue, palmatine, purpurogallin, theaflavin monogallates, and thimerosal. IC50 shown for those fitting 4 parameter linear regression curve.

FIG. 4 shows the fluorescence polarization orthogonal screen results. MSUT2-bound FAM-poly(A)RNA emits highly polarized light, while inhibition results in free FAM-poly(A) and emits non-polarized light (Baker et. al). Y axis is polarization units (mP) and X axis is compound concentration in μM. FAM-labeled RNA IC50 indicated for all compounds.

FIG. 5 shows a cellular toxicity assay. Cell viability measured by cell-titer glo (Promega) for 4 different compound treatments at indicated concentrations and normalized to control. Dashed line indicates control viability. Error bars represent standard error of the mean.

FIGS. 6A-B show the Selectivity screen. Compounds were screened by Alpha Screen (FIG. 6A) for MSUT2 and PABPN1 (FIG. 6B) dose response. IC50s indicated. Ebselen exhibits 4.6 fold selectivity for MSUT2 over PAPBN1.

FIGS. 7A-E show the underlying biology and validation for the fluorescence polarization assay used to identify inhibitory compounds. FIG. 7A shows a schematic of MSUT2 (ZC3H14). Targeted construct consisted of the c-terminal end (dark blue and amino acids 601-736) of MSUT2. 5 CCCH finger domains are indicated (light blue). FIG. 7B shows fluorescence polarization depicting MSUT2 (dark blue) bound to FAM labeled RNA emitting highly polarized light and free FAM-RNA emitting low levels of polarized light after disruption by inhibitor. FIG. 7C shows the saturation assay holding FAM-RNA concentration constant at 10 nM and increasing concentrations of MSUT2. FIG. 7D shows the competition assay with MSUT2 concentration at 125 nM and FAM-RNA at 10 nM with increasing concentrations of unlabeled poly(A)₁₅. FIG. 7E shows a Z′-factor bar graph showing negative controls on the left and positive controls on the right. Three standard deviations on either side of the means are indicated and Y-axis values are Polarization values in units of mP. Z′-factor determined to be 0.748. Signal to background ratio was determined to be 69.6.

FIG. 8 shows a workflow schematic for drug selection. The NIH Clinical Collection was first screened by fluorescence polarization followed by a battery of secondary validation measures including dose-response analysis, specificity counter-screening against PABPN1, and replication through Alpha Assay orthogonal screening. Compounds passing initial filters were then subjected to a cellular toxicity assay and assessed for physiological effect on tau protein using a human cell model of tau aggregation. Initial hits from the primary screen were validated by FP dose-response characterization.

FIGS. 9A-B show the screening and identified hits for the National Institutes of Health Clinical Collection. FIG. 9A shows a graph depicting fluorescence polarization screen of NIH Clinical Collection compounds at 10 μM. Hit window (blue) began below −4σ (standard deviations) from the mean and included 12 primary hits. FIG. 9B shows structures of compounds identified through primary screen.

FIG. 10 shows a dose response by fluorescence polarization of eight positive hits with calculated IC50s indicated as determined by 4-parameter non-linear regression.

FIG. 11 shows the PABPN1 fluorescence polarization counter screen for compounds which passed dose-response filter IC50s indicated as determined by 4-parameter non-linear regression.

FIG. 12 shows the Alpha Assay orthogonal dose-response validation where Y-axis is Alpha Count for compounds which showed dose-response activity. IC50s indicated and determined by 4-parameter non-linear regression.

FIG. 13 shows the cell viability assay (Promega Cell Titer Glo) for three validated compounds at indicated concentrations. Dashed line represents 100% normalized viability for 2% DMSO vehicle.

### DETAILED DESCRIPTION

Many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

As used in the specification and in the claims, the term “comprising” can include the aspects “consisting of” and “consisting essentially of” “Comprising” can also mean “including but not limited to.”

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

The word “or” as used herein means any one member of a particular list and also includes any combination of members of that list.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for Alzheimer's disease, a taupathy disorder or dementia, such as, for example, prior to the administering step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. Treatment can also be administered to a subject to ameliorate one more signs of symptoms of a disease, disorder, and/or condition. For example, the disease, disorder, and/or condition can be relating to Alzheimer's disease, Alzheimer's disease-related dementia or dementia or a taupathy disorder.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

Tauopathies are neurological disorders characterized by intracellular tau deposits forming neurofibrillary tangles, neuropil threads, or other disease-specific aggregates composed of the protein tau. Tauopathy disorders include frontotemporal lobar degeneration, corticobasal degeneration, Pick's disease, and the largest cause of dementia, Alzheimer's disease. The lack of disease-modifying therapeutic strategies to address tauopathies remains an important unmet need in dementia care. Thus, new broad spectrum tau targeted therapeutics could have profound impact in multiple tauopathy disorders including Alzheimer's disease. Disclosed herein is a drug-discovery paradigm to identify inhibitors of the pathological tau enabling protein, MSUT2. It has been shown that the activity of the RNA binding protein MSUT2 drives tauopathy including tau-mediated neurodegeneration and cognitive dysfunction in mouse models. Thus, it was tested whether MSUT2 inhibitors could be therapeutic for tauopathy disorders. The idea that new therapeutics against targets controlling tau deposition could potentially impact several disorders as tauopathies share common pathology. Disclosed herein is a MSUT2 inhibiting tool compound identification method that includes a primary Alpha Screen. In some aspects, this method can be followed by dose-response validation, a secondary fluorescence polarization orthogonal assay, a tertiary specificity screen, and a preliminary toxicity screen. The findings disclosed herein serve as a proof of principle methodology for finding specific inhibitors of the RNA binding protein MSUT2:poly(A) RNA interaction and resulted in the identification of Ebselen as a potential tool compound.

It has been difficult to target tau directly; however, recent work has provided a role for the RNA-binding protein MSUT2 in exacerbating the development of toxic tau aggregates (Guthrie, C. R., et al., Hum Mol Genet 2011, 20 (10), 1989-99). In a mouse model of tauopathy (PS19), MSUT2 overexpression induces pathological tau deposition, widespread hippocampal neuron loss, as well as deficits in cognition (Wheeler, J. M., et al., Science Translational Medicine 2019, 11 (523)). Knockout of MSUT2 is innocuous, but leads to preservations of neurons and cognition by reducing tangle formation and neurofibrillary degeneration. MSUT2 binds poly(A) RNA and plays a role in mRNA transcript maturation (Brockmann, C., et al., Structure 2012, 20 (6), 1007-1018; Wigington, C. P., et al., Wiley Interdiscip Rev RNA 2014, 5 (5), 601-22; and Rha, J., et al., Hum Mol Genet 2017, 26 (19), 3663-3681). As disclosed herein, it was tested whether targeting the poly(A):MSUT2 interaction with small molecules will reduce toxic tau burden and may be a viable therapeutic approach for tauopathies.

MSUT2 belongs to a class of proteins known as RNA binding proteins (RBPs). Considerable evidence has implicated many different RBPs in diverse neurodegenerative diseases. The early-onset neurodegenerative disorder spinal muscular atrophy (SMA) is caused by loss-of-function mutations in Survival of motor neuron 1 (SMN1) gene (Perego, M. G. L., et al., Cell Mol Life Sci 2020). SMN1 encodes the SMN protein important for transcriptional regulation and mutations disrupt cause widespread disruption in splicing homeostasis and is responsible for SMA pathology (Groen, E. J. N., et al., Nature Reviews Neurology 2018, 14 (4), 214-224). Gene therapy targeting SMN1, onasemnogene abeparvovec, has been a tremendous success story in treating RBP-mediated neurodegeneration (Al-Zaidy, S. A., et al., J Neuromuscul Dis 2019, 6 (3), 307-317). Other RBPs implicated in neurodegeneration include TDP-43 in Amyotrophic Lateral Sclerosis (ALS) and frontotemporal lobar dementia (Taylor, J. P., et al., Nature 2016, 539 (7628), 197-206; Neumann, M., et al., Science 2006, 314 (5796), 130-3), FUS in ALS (Shang, Y., et al., Brain Res 2016, 1647, 65-78), PABPN1 in oculopharyngeal muscular dystrophy (Malerba, A., et al., Nature Communications 2017, 8 (1), 14848), and PARK7 (DJ-1) in Parkinson's disease among many others (Bonifati, V., et al., Science 2003, 299 (5604), 256-9; Conlon, E. G., et al., Genes Dev 2017, 31 (15), 1509-1528; and Ito, D., et al., Science Translational Medicine 2017, 9 (415), eaah5436).

To date there are no clinically-approved therapeutics for the treatment of tauopathies and the approved treatment modalities, including cholinesterase inhibitors, are limited to helping to ameliorate symptoms (Brunden, K. R., et al., Nature Reviews Drug Discovery 2009, 8 (10), 783-793; and Rojas, J. C., et al., Nature Reviews Neurology 2016, 12 (2), 74-76). However, there is an ongoing initiative to develop tau-modifying therapeutics which target tau propagation, tau aggregation, tau levels, and tau PTMs among others (Congdon, E. E., et al., Nature Reviews Neurology 2018, 14 (7), 399-415; and Li, C., et al., Nature Reviews Drug Discovery 2017, 16 (12), 863-883). Targeting MSUT2 presents a challenge because no solved protein structure exists and the precise pathological mechanism of MSUT2 molecular action in tauopathies remains unclear. Further, MSUT2 exhibits no enzymatic activity and its interaction with poly(A) likely comprises a relatively large interacting surface area typical of most RNA binding proteins (Stefl, R., et al., EMBO Rep 2005, 6 (1), 33-38; and Dominguez, D., et al., Molecular Cell 2018, 70 (5), 854-867). MSUT2 also interacts with an important regulator of mRNA poly(A) tails, PABPN1. Thus, therapeutic strategies must specifically target MSUT2 without disrupting PABPN1 function because PABPN1 is important. Finally, the potentially challenging nature of RBPs as screening targets have resulted in relatively few small-molecule screening initiatives to date.

Despite these challenges, described herein are compositions and methods that can be used to identify small molecules which specifically disrupt poly(A):MSUT2 interaction. The methods include incorporating one or more of the following: a primary Alpha Screen, dose response validation, orthogonal FP validation, a specificity counter-screen against PABPN1, and a cellular toxicity screen. As described herein the Spectrum Collection was screened as a proof-of-principle application of a discovery pipeline and identified Ebselen as a potential tool compound. Ebselen has been shown to have broad spectrum application as a general anti-inflammatory and may have repurposing potential as an MSUT2 inhibitor.

Compositions

Disclosed herein are compositions comprising one or more of the mammalian suppressor of tauopathy 2 (MSUT2) inhibitors listed in Table 1.

TABLE 1
MSUT2 inhibitors.

| MSUT2 | | | Ki (nm) for |
|---|---|---|---|
| Inhibitor | Known Target | Ki (nM) for Known Target | MSUT2 |
| Duloxetine | Sodium-dependent | 0.501, 0.8, 4.6, 5, 790 | 279 |
| | serotonin transporter | 16, 45, 5.97, 7.5, 7.94, 7450 | |
| | Sodium-dependent | drugbank.ca/drugs/DB00476 | |
| | noradrenaline | | |
| | transporter | | |
| Saquinavir | Human | 13.0, 9.0, 0.51, 12.0, 0.22, 71.0 | 188 |
| | immunodeficiency virus | ebi.ac.uk/chembl/compound_re | |
| | mtype 1 protease | port_card/CHEMBL114/ | |
| Clofazimine | Mycobacterium leprae | MIC (minimum inhibitory | 978 |
| | DNA | concentration) unavailable | |
| | | against M. leprae | |
| | | ebi.ac.uk/chembl/compound_re | |
| | | port_card/CHEMBL1292/ | |
| Hydroxyzine | Histamine H1 Receptor | 1, 2 | 573 |
| (cetirizine) | | (drugbank.ca/drugs/DB00557) | |
| Atomoxetine | Norepinephrine | 0.7, 5.0 | failed dose |
| | transporter | ebi.ac.uk/chembl/compound_re | response (DR) |
| | | port_card/CHEMBL641/ | |
| Dipyridamole | 3′,5′-cyclic | 1000 | failed DR |
| | phosphodiesterase | drugbank.ca/drugs/DB00975 | (metal ion |
| | Equilibrative nucleoside | 8.18, 8.79 | binding, zinc |
| | transporter 1 | drugbank.ca/drugs/DB00975 | ion binding) |
| Chloramphenicol | Bacterial 70S ribosome | MIC: Large range of values | failed DR |
| | | (ebi.ac.uk/chembl/compound_re | |
| | | port_card/CHEMBL130/); | |
| | | difficult to compare organismal | |
| | | to MSUT2 targeting | |
| Nafadotride (not | Dopamine D3 Receptor | 0.11, 0.81 | 867 |
| an approved | | ebi.ac.uk/chembl/compound_re | |
| drug) | | port_card/CHEMBL286252/ | |
| Indinavir | Human | 0.31, 50, 40, 0.07, 15.0, 0.52, | 916 |
| | immunodeficiency virus | 0.37, 0.07, 0.6, 0.8, 10.4, 2.51. | |
| | type 1 protease | 0.37, 0.24, 0.31, 1.34, 0.37, | |
| | | 0.28, 7.0, 0.07 | |
| | | ebi.ac.uk/chembl/compound_re | |
| | | port_card/CHEMBL115/ | |
| Granisetron | Serotonin 3a (5-HT3a) | 1.45, 1.45, 3.981 | 534 |
| | receptor | ebi.ac.uk/chembl/compound_re | |
| | | port_card/CHEMBL289469/ | |
| Flurbiprofen | Prostaglandin G/H | 4230, 5500, 770 | 2460 |
| | synthase | drugbank.ca/drugs/DB00712 | |
| | 2(drugbank.ca/drugs/DB | | |
| | 00712) | | |
| Zeranol (not | | Non-inhibitory (synthetic | failed DR |
| approved in | | nonsteroidal estrogen); zinc ion | |
| humans, only | | binding molecule | |
| cattle) | | | |
| Ebselen (also | Soluble epoxide | 0.550 (PMID: 23219563) | 1.019 μM by |
| called PZ 51, | hydrolase (gene | A synthetic organoselenium | FP assay; 1.58 |
| DR3305, and | EPXH2; drugbank) | drug molecule with anti- | μM by Alpha |
| SPI-1005) | Acts as a mimic of | inflammatory, anti-oxidant and | assay |
| | glutathione peroxidase | cytoprotective activity | |
| | and can also react with | | |
| | peroxynitrite. Is a | | |
| | scavenger of hydrogen | | |
| | peroxide as well as | | |
| | hydroperoxides | | |
| | including membrane | | |
| | bound phospholipid and | | |
| | cholesterylester | | |
| | hydroperoxides. Also is | | |
| | a selective inhibitor of | | |
| | glucose-6-phosphate | | |
| | isomerase (CpGPI) | | |

Ki for the “MSUT2” was calculated using Cheng Prusof equation: Ki=(IC50)/[(L/Kd)+1]. There are many Ki, IC50, MIC, and potency studies with wide-ranging values depending on the assay. These studies can be found at ChEMBL by searching the compound, then clicking on “Activity Charts” on the right menu. ebi.ac.uk/chembl/compound_report_card/CHEMBL1175/. Alternatively, Kis can be retrieved from drugbank.ca.

ChEMBL used for the compounds listed in Table 1. Note that in some cases two more targets have been described; the target listed is the primary target found by searching the compound on ChEMBL and clicking on “drug mechanism” on the right menu.

In some aspects, the one or more MSUT2 inhibitors can be duloxetine, saquinavir or clofazimine or an analog thereof. In some aspects, the MSUT2 inhibitor can be ebselen or an analog thereof such as those described in Satheeshkumar K, Mugesh G (2011), Chem. Eur. J. 17 (17): 4849-57, which is hereby incorporated by reference in its entoirity for its teaching of ebselen analogs. In some aspects, duloxetine, saquinavir or clofazimine can have a Ki lower for MSUT2 than for its known target thereby allowing a lower therapeutically effective amount to be administered.

Any of the compositions disclosed herein can further comprise a pharmaceutically acceptable carrier. In some aspects, the pharmaceutically acceptable carrier can be buffered saline, water or DMSO. In some aspects, the pharmaceutically acceptable carrier can comprise a lipid-based or polymer-based colloid. In some aspects, the colloid can be a liposome, a hydrogel, a microparticle, a nanoparticle, or a block copolymer micelle. In some aspects, the compositions described herein can be formulated for intravenous, subcutaneous, intrathecal, intramuscular, oral, intranasal, local or direction injection or intraperitoneal administration.

In some aspects, any of the compositions disclosed herein (or composition comprising any of the MSUT2 inhibitors) can reduce accumulation of phosphorylated and aggregated human tau protein in a subject. In some aspects, the subject has Alzheimer's disease, a tauopathy disorder or dementia.

Methods of Treatment

The methods disclosed herein can be useful for the treatment of a subject with Alzheimer's disease, a tauopathy disorder or dementia. The method can comprise administering to a subject with Alzheimer's disease, a tauopathy disorder or dementia a therapeutically effective amount of one or more of the mammalian suppressor of tauopathy 2 (MSUT2) inhibitors listed in Table 1. In some aspects, the MSUT2 inhibitor can be ebselen. In some aspects, the therapeutically effective amount can reduce accumulation of phosphorylated and aggregated human tau.

The methods disclosed herein can be useful for inhibiting expression of a MSUT2 polynucleotide. In some aspects, the method can inhibit expression of a MSUT2 polynucleotide in a subject. The method can comprise administering to a subject with Alzheimer's disease, a tauopathy disorder or dementia a therapeutically effective amount of one or more of the mammalian suppressor of tauopathy 2 (MSUT2) inhibitors listed in Table 1. In some aspects, the method can comprise contacting a cell with one or more of the MSUT2 inhibitors listed in Table 1. In some aspects, the suppressor of MSUT2 can reduce accumulation of phosphorylated and aggregated tau. In some aspects, the expression of the MSUT2 polynucleotide can be inhibited or suppressed by one or more of the MSUT2 inhibitors listed in Table 1. In some aspects, the one or more of the MSUT2 inhibitors listed in Table 1 can inhibit the binding of poly(A) RNA to the MSUT2 polynucleotide. In some aspects, the cell can be a eukaryotic cell. In some aspects, the cell can be a mammalian cell. In some aspects, the mammalian cell can be a brain cell. In some aspects, the cell can be in an individual. In some aspects, the individual can be a human.

The methods disclosed herein can be useful for reducing phosphorylated and aggregated human tau protein in a subject. The methods can comprise administering to a subject with Alzheimer's disease, a tauopathy disorder or dementia a therapeutically effective amount of one or more of the mammalian suppressor of tauopathy 2 (MSUT2) inhibitors listed in Table 1.

The methods disclosed herein can be useful for suppressing expression of a MSUT2 polynucleotide. In some aspects, the method can suppress expression of a MSUT2 polynucleotide in a subject. The method can comprise administering to a subject with Alzheimer's disease, a tauopathy disorder or dementia a therapeutically effective amount of one or more of the mammalian suppressor of tauopathy 2 (MSUT2) inhibitors listed in Table 1. In some aspects, the method can comprise contacting a cell with one or more of the MSUT2 inhibitors listed in Table 1. In some aspects, the suppressor of MSUT2 can reduce accumulation of phosphorylated and aggregated tau. In some aspects, the expression of the MSUT2 polynucleotide can be inhibited or suppressed by the one or more of the MSUT2 inhibitors listed in Table 1. In some aspects, the one or more of the MSUT2 inhibitors listed in Table 1 can inhibit the binding of poly(A) RNA to the MSUT2 polynucleotide. In some aspects, the cell can be a eukaryotic cell. In some aspects, the cell can be a mammalian cell. In some aspects, the mammalian cell can be a brain cell. In some aspects, the cell can be in an individual. In some aspects, the individual can be a human.

The methods disclosed herein can be useful for potentiating a neuroinflammatory response to a pathological tau protein. In some aspects, the method can potentiate a neuroinflammatory response to a pathological tau protein in a subject. The method can comprise administering to a subject with Alzheimer's disease, a tauopathy disorder or dementia a therapeutically effective amount of one or more of the mammalian suppressor of tauopathy 2 (MSUT2) inhibitors listed in Table 1. In some aspects, the methods can comprise contacting a cell with one or more of the MSUT2 inhibitors listed in Table 1. In some aspects, the suppressor of tauopathy 2 (MSUT2) can reduce accumulation of phosphorylated and aggregated tau. In some aspects, the cell can be a eukaryotic cell. In some aspects, the cell can be a mammalian cell. In some aspects, the mammalian cell can be a brain cell. In some aspects, the cell can be in an individual. In some aspects, the individual can be a human.

The methods disclosed herein can be useful for decreasing astrocytosis or microgliosis. In some aspects, the method can decrease astrocytosis or microgliosis in a subject. The method can comprise administering to a subject with Alzheimer's disease, a tauopathy disorder or dementia a therapeutically effective amount of one or more of the mammalian suppressor of tauopathy 2 (MSUT2) inhibitors listed in Table 1. In some aspects, the method can comprise contacting a cell with one or more of the MSUT2 inhibitors. In some aspects, the one or more of the MSUT2 inhibitors listed in Table 1 can reduce accumulation of phosphorylated and aggregated tau. In some aspects, the cell can be a eukaryotic cell. In some aspects, the cell can be a mammalian cell. In some aspects, the mammalian cell can be a brain cell. In some aspects, the cell can be in an individual. In some aspects, the individual can be a human.

The methods disclosed herein can be useful for reducing neuroinflammation. In some aspects, the method can reduce neuroinflammation in a subject. The method can comprise administering to a subject with Alzheimer's disease, a tauopathy disorder or dementia a therapeutically effective amount of one or more of the mammalian suppressor of tauopathy 2 (MSUT2) inhibitors listed in Table 1. In some aspects, the method can comprise contacting a cell with one or more of the MSUT2 inhibitors listed in Table 1. In some aspects, one or more of the MSUT2 inhibitors listed in Table 1 can reduce accumulation of phosphorylated and aggregated tau. In some aspects, the expression of the MSUT2 polynucleotide can be inhibited or suppressed by the one or more of the MSUT2 inhibitors listed in Table 1. In some aspects, the one or more of the MSUT2 inhibitors listed in Table 1 can inhibit the binding of poly(A) RNA to the MSUT2 polynucleotide. In some aspects, the cell can be a eukaryotic cell. In some aspects, the cell can be a mammalian cell. In some aspects, the mammalian cell can be a brain cell. In some aspects, the cell can be in an individual. In some aspects, the individual can be a human.

In some aspects, the one or more MSUT2 inhibitors can be duloxetine, saquinavir or clofazimine. In some aspects, the one or more MSUT2 inhibitors can be duloxetine, saquinavir or clofazimine. In some aspects, the MSUT2 inhibitor can be ebselen. In some aspects, duloxetine, saquinavir or clofazimine can have a Ki lower for MSUT2 than for its known target thereby allowing a lower therapeutically effective amount to be administered.

In some aspects, the subject has Alzheimer's disease. In some aspects, the subject has dementia. In some aspects, the subject has mild-moderate Alzheimer's disease. In some aspects, the subject has moderate-severe Alzheimer's disease. Alzheimer's disease typically progresses slowly in three general stages, mild (early stage), moderate (middle stage) and severe (late stage). In mild Alzheimer's disease (early stage), subjects can still function independently but may notice that they are having memory lapses such as forgetting familiar words or the location of everyday objects. During moderate Alzheimer's disease (middle stage), subjects may have greater difficulty performing tasks (e.g., paying bills) and confusing words, but may still remember significant details about their life. In addition, subjects in this stage may feel moody or withdrawn, are at an increased risk of wandering and becoming lost, and can exhibit personality and behavioral changes including suspiciousness and delusions or compulsive, repetitive behavior. In severe Alzheimer's disease (late stage), subjects lose the ability to respond to their environment, to carry on a conversation and eventually, to control movement. Also, during this severe stage, subjects need extensive help with daily activities and have increasing difficulty communicating. In some aspects, the subject has an Alzheimer's-related dementia. In some aspects, the Alzheimer's-related dementia can be progressive supranuclear palsy, chronic traumatic encephalopathy, frontotemporal lobar degeneration, or other tauopathy disorders. In some aspects, the subject has a tauopathy disorder. The methods disclosed herein can be effective for targeting one or more genes, including mammalian suppressor of tauopathy 2 (MSUT2). In some aspects, the methods also include the step of administering a therapeutic effective amount of one or more MSUT2 inhibitors listed in Table 1.

In some aspects, the methods of treating a subject can comprise contacting a cell or a subject with an effective amount with one or more of the MSUT2 inhibitors listed in Table 1. In some aspects, the cell can be a vertebrate, a mammalian or a human cell. In some aspects, the cell can be a eukaryotic cell. In some aspects, the cell can be a brain cell. In some aspects, the cell can be in an individual. In some aspects, the individual can be a human.

In some aspects, the MSUT2 inhibitor listed in Table 1 can potentiate the neuroinflammatory response to pathological tau. In some aspects, the MSUT2 inhibitor listed in Table 1 can decrease astrocytosis and microgliosis.

In some aspects, the methods can further include the step of identifying a subject (e.g., a human patient) who has Alzheimer's disease, a tauopathy disorder or dementia and then providing to the subject a composition comprising the one or more of the MSUT2 inhibitors as disclosed herein. In some aspects, the subject has an Alzheimer's-related dementia. In some aspects, the Alzheimer's-related dementia can be progressive supranuclear palsy, chronic traumatic encephalopathy, frontotemporal lobar degeneration, or other tauopathy disorders. In some aspects, the subject can be identified using standard clinical tests known to those skilled in the art. While a definite AD diagnosis requires post-mortem examination, skilled clinicians can conduct an evaluation of cognitive function with over 95% accuracy. Examples of tests for diagnosing Alzheimer's disease or dementia include Mini-Mental State Examination (MMSE), Mini-cog© Score, Alzheimer's Disease Composite Score (ADCOMS), Alzheimer's Disease Assessment Scale-cognitive subscale (ADAS-Cog) and Clinical Dementia Rating Sum of Boxes (CDR-SB).

In some aspects, the tauopathy disorder can be a degenerative disorder. Examples of tauopathy disorders include but are not limited to primary tauopathies (e.g., Frontotemporal Lobar Degeneration Frontotemporal Dementia (FTLD), primary progressive aphasia, including atypical dopaminergic-resistant Parkinsonian syndromes with prominent extra-pyramidal symptoms and corticobasal syndrome); secondary tauopathies; Pick disease; progressive supranuclear palsy; corticobasal degeneration; argyrophilic grain disease; globular glial tauopathies; and primary age-related tauopathy, which includes neurofibrillary tangle dementia, chronic traumatic encephalopathy (CTE), and aging-related tau astrogliopathy.

The therapeutically effective amount can be the amount of the composition administered to a subject that leads to a full resolution of the symptoms of the condition or disease, a reduction in the severity of the symptoms of the condition or disease, or a slowing of the progression of symptoms of the condition or disease. The methods described herein can also include a monitoring step to optimize dosing. The compositions described herein can be administered as a preventive treatment or to delay or slow the progression of degenerative changes.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with Alzheimer's disease, a tauopathy disorder, or dementia in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the disease, disorder or condition (e.g., Alzheimer's disease, a tauopathy disorder, or dementia) is delayed, hindered, or prevented, or the disease, disorder or condition (e.g., Alzheimer's disease, a tauopathy disorder, or dementia) or a symptom of the disease, disorder or condition (e.g., Alzheimer's disease, a tauopathy disorder, or dementia) is ameliorated. One or more of the symptoms can be less severe. Recovery can be accelerated in an individual who has been treated.

The compositions disclosed herein can be used in a variety of ways. For instance, the compositions disclosed herein can be used for direct delivery of modified therapeutic cells. The compositions disclosed herein can be used or delivered or administered at any time during the treatment process. The compositions described herein including cells or a virus can be delivered to the one or more brain regions, one or more brain cells, or to brain regions or brain cells to stop or prevent one or more signs of symptoms of the disease or condition in an adjacent brain region or brain cell.

The dosage to be administered depends on many factors including, for example, the route of administration, the formulation, the severity of the patient's condition/disease, previous treatments, the patient's size, weight, surface area, age, and gender, other drugs being administered, and the overall general health of the patient including the presence or absence of other diseases, disorders or illnesses. The particular dosage of a pharmaceutical composition to be administered to the patient will depend on a variety of considerations (e.g., the severity of the symptoms of the disease, disorder or condition), the age and physical characteristics of the subject and other considerations known to those of ordinary skill in the art. Variations in the needed dosage may be expected. Variations in dosage levels can be adjusted using standard empirical routes for optimization. Dosages can be established using clinical approaches known to one of ordinary skill in the art. Administrations of the compositions described herein can be single or multiple (e.g., 2- or 3-, 4-, 6-, 8-, 10-, 20-, 50-, 100-, 150-, or more fold). Further, encapsulation of the compositions in a suitable delivery vehicle (e.g., polymeric microparticles or implantable devices) can improve the efficiency of delivery. In some aspects, a dose can comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In some aspects of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described herein. The foregoing doses include amounts between those indicated and are intended to also include the lower and upper values of the ranges. The practitioner responsible for administration can, in any event, determine the concentration of active ingredient(s) in a composition and appropriate dose(s) for the individual subject.

The therapeutically effective amount of the compositions described herein can include a single treatment or a series of treatments (i.e., multiple treatments or administered multiple times). Treatment duration using any of compositions disclosed herein can be any length of time, such as, for example, one day to as long as the life span of the subject (e.g., many years). For instance, the composition can be administered daily, weekly, monthly, yearly for a period of 5 years, ten years, or longer. The frequency of treatment can vary. For example, the compositions described herein can be administered once (or twice, three times, etc.) daily, weekly, monthly, or yearly for a period of 5 years, ten years, or longer.

In some aspects, the compositions disclosed herein can also be co-administered with another therapeutic agent. In some aspects, the methods disclosed herein can further comprise administering a cholinesterase inhibitor to the subject. In some aspects, the cholinesterase inhibitor can be galantamine, rivastigmine or donepezil. In some aspects, the methods disclosed herein can further comprise administering an anti-inflammatory therapy to the subject.

In some aspects, the methods disclosed herein also include treating a subject having Alzheimer's disease, a tauopathy disorder or dementia. In some aspects, the methods disclosed herein can include the step of determining MSUT2 levels in a subject. In some aspects, the disclosed methods can further include the step of administering to the subject a pharmaceutical composition comprising a one or more of the MSUT2 inhibitors listed in Table 1.

Pharmaceutical Compositions

As disclosed herein, are pharmaceutical compositions, comprising the compositions disclosed herein. In some aspects, the pharmaceutical composition can comprise any of MSUT2 inhibitors disclosed herein. For example, disclosed herein are pharmaceutical compositions comprising one or more of the MSUT2 inhibitors listed in Table 1. In some aspects, the pharmaceutical compositions further comprise a pharmaceutically acceptable carrier.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration, and can include one or more physiologically acceptable excipients, which can vary depending on the route of administration. Any of the MSUT2 inhibitors or other drugs described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions as disclosed herein can be prepared for oral or parenteral administration. Pharmaceutical compositions prepared for parenteral administration include those prepared for intravenous (or intra-arterial), intramuscular, subcutaneous, intrathecal, transmucosal (e.g., intranasal) direct or local injection, transdermal (e.g., topical) or intraperitoneal administration. Aerosol inhalation can also be used. Paternal administration can be in the form of a single bolus dose, or may be, for example, by a continuous pump. In some aspects, the local or direct injection can be via convection enhanced delivery. In some aspects, the compositions can be prepared for parenteral administration that includes dissolving or suspending any of the MSUT2 inhibitors disclosed herein in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

In some aspects, the compositions disclosed herein are formulated for oral, intramuscular, intravenous, subcutaneous, intrathecal, direct or local injection, intranasal, or intraperitoneal administration.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. As used herein “pharmaceutically acceptable” means molecules and compositions that do not produce or lead to an untoward reaction (i.e., adverse, negative or allergic reaction) when administered to a subject as intended (i.e., as appropriate).

In some aspects, the compositions as disclosed herein can be delivered to a cell of the subject. In some aspects, such action can be achieved, for example, by using polymeric, biodegradable microparticle or microcapsule delivery vehicle, sized to optimize phagocytosis by phagocytic cells (e.g., macrophages).

The compositions as disclosed herein can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery. In some aspects, the route of administration includes but is not limited to direct injection into the brain. Such administration can be done without surgery, or with surgery.

Methods of Screening

Disclosed herein are methods of screening for compounds capable of inhibiting MSUT2 binding to poly(A) RNA. In some aspects, the method can comprise contacting at least one candidate compound, poly(A) RNA and polyadenylate-binding nuclear protein 1 (PABPN1) under conditions in which PABPN1 is capable of stimulating RNA polyadenylation in the absence of the candidate compound. In some aspects, the method can comprise determining whether the candidate compound inhibits MSUT2 binding to poly(A) RNA. In some aspects, the method can comprise selecting the candidate compound which inhibits MSUT2 binding to poly(A) RNA. In some aspects, the inhibition of MSUT2 binding to poly(A) RNA can be measured by RNA polyadenylation. In some aspects, the candidate compound selected can inhibit formation of a macromolecular complex. In some aspects, the macromolecular complex can comprise MSUT2, PABPN1 and poly(A) RNA. In some aspects, the method further comprises purifying the candidate compound can be purified. In some aspects, the method further comprises isolating the candidate compound can be isolated.

Disclosed herein are methods for screening compounds for pharmacological intervention in one or more tauopathy disorders. In some aspects, the method can comprise providing an assay for MSUT2 to bind to poly(A) RNA and its modulation of RNA polyadenylation. In some aspects, the method can comprise providing a purified or non-purified compound or purified or non-purified mixture of compounds. In some aspects, the method can comprise screening the purified or non-purified compound or purified or non-purified mixture of compounds in an environment that can allow for inhibition of MSUT2 binding to poly(A) RNA by the purified or non-purified compound or purified or non-purified mixture of compounds in the assay. In some aspects, the method can comprise isolating the one or more compounds that inhibit MSUT2 binding to poly(A) RNA. In some aspects, the method can comprise the inhibition of MSUT2 binding to poly(A) RNA is measured by RNA polyadenylation. In some aspects, the assay can comprise forming a macromolecular complex that can comprise MSUT2, PABPN1, and poly(A) RNA.

In some aspects, the tauopathy disorder can be a degenerative disorder. Examples of tauopathy disorders include but are not limited to primary tauopathies (e.g., Frontotemporal Lobar Degeneration Frontotemporal Dementia (FTLD), primary progressive aphasia, including atypical dopaminergic-resistant Parkinsonian syndromes with prominent extra-pyramidal symptoms and corticobasal syndrome); secondary tauopathies; Pick disease; progressive supranuclear palsy; corticobasal degeneration; argyrophilic grain disease; globular glial tauopathies; and primary age-related tauopathy, which includes neurofibrillary tangle dementia, chronic traumatic encephalopathy (CTE), and aging-related tau astrogliopathy.

Disclosed herein are methods comprising an alpha screen. Disclosed herein are methods of detecting RNA polyadenylation of poly(A) RNA. In some aspects, the method can comprise: a) providing at least one candidate compound; b) providing at least one poly(A) RNA molecule bound to at least one donor component, wherein the at least one donor component emits a signal after it is irradiated by a light source; c) providing at least one MSUT2 polypeptide bound to at least one acceptor component, wherein the at least one acceptor component is able to receive a signal from the at least one donor component and emit a signal in the form of an electromagnetic radiation; d) bringing the at least one candidate compound, the at least one poly(A) RNA molecule bound to at least one donor component; and the at least one MSUT2 polypeptide bound to at least one acceptor component in contact with each other; e) irradiating the mixture of step d) with a light source; and f) measuring the electromagnetic radiation emitted by the mixture.

In some aspects, a signal can be emitted from the at least one acceptor component when the at least one donor component and the at least one acceptor component are within close proximity of one another indicating that the candidate compound does not inhibit RNA polyadenylation of the poly(A) RNA, and thereby does not inhibit MSUT2 polypeptide binding to poly(A) RNA.

In some aspects, a signal can be emitted from the at least one donor component when the at least one donor component and the at least one acceptor component are not within close proximity of one another indicating that the candidate compound inhibits RNA polyadenylation of the poly(A) RNA, and thereby inhibits MSUT2 polypeptide binding to poly(A) RNA.

In some aspects, the signal can be transferred from the donor component to the acceptor component by emissionless energy transfer. In some aspects, the emissionless energy transfer can be fluorescence resonance energy transfer. In some aspects, the signal can be transferred from the donor component to the acceptor component via singlet oxygen wherein the donor component is capable of converting triplet oxygen to singlet oxygen after excitation by a laser and wherein the acceptor component is excitable by the singlet oxygen and capable of absorbing in an emissionless manner and emitting in the form of fluorescence radiation the energy absorbed. In some aspects, the candidate compound identified can be purified. In some aspects, the candidate compound identified can be isolated.

In some aspects, the poly(A) RNA molecule can be linked to a biotin molecule and the donor component can be linked to streptavidin. In some aspects, the MSUT2 polypeptide can be a recombinant zinc finger protein. In some aspects, the recombinant MSUT2 zinc finger protein can be linked to gluthathione S-transferase and the acceptor component can be linked to glutathione. In some aspects, the electromagnetic radiation can be fluorescence radiation. In some aspects, the light source can be a laser.

Disclosed herein are methods comprising a fluorescence polarization/anisotropy screen. Disclosed herein are methods for identifying a candidate composition capable of inhibiting a RNA binding protein (RBP) binding to poly(A) RNA. In some aspects, the method can comprise: a) contacting a fluorescent probe molecule bound to the poly(A)RNA molecule (FAM-RNA), the RBP, and the candidate composition in a sample under conditions in which the RBP is capable of binding to the FAM-RNA molecule and forming a macromolecular complex, wherein the macromolecular complex comprises FAM-RNA:RBP; b) exciting fluorescence in the sample with linearly polarized light from a pulsed excitation source; c) detecting a fluorescent emission from the excited sample; and d) measuring anisotropy of the emitted fluorescence, wherein the reduction of emitted polarized fluorescence identifies a candidate composition capable of inhibiting a RNA binding protein (RBP) binding to poly(A) RNA. In some aspects, RBP+PolyA=high emitted fluorescence polarization. In some aspects, RBP (inhibitor) PolyA=low emitted fluorescence polarization. In some aspects, an effective compound can reduce detected polarized fluorescence.

In some aspects, the RBP can be a recombinant MSUT2 zinc finger protein. In some aspects, the RBP can be PABPN1. In some aspects, the method can further comprise selecting the candidate compound which inhibits the formation of the macromolecular complex. In some aspects, the candidate compound which inhibits the formation of the macromolecular complex inhibits RBP binding to FAM-RNA, wherein the FAM-RNA emits a low level of polarized light. In some aspects, the macromolecular complex emits a high level of polarized light. In some aspects, the anisotropy of the emitted fluorescence is by determining the intensities of fluorescent emission of the FAM-RNA and/or the macromolecular complex.

In some aspects, the methods of identifying a MSUT2 inhibitor can comprise performing the alpha screen described herein. In some aspects, the methods of identifying a MSUT2 inhibitor can comprise performing the fluorescence polarization/anisotropy screen described herein. In some aspects, the method of identifying a MSUT2 inhibitor can comprise performing the alpha screen described herein, the fluorescence polarization/anisotropy screen described herein, or a combination thereof. In some aspects, the method of identifying a MSUT2 inhibitor can comprising performing the alpha screen described herein followed by performing the fluorescence polarization/anisotropy screen described herein. In some aspects, the method of identifying a MSUT2 inhibitor can comprising performing the fluorescence polarization/anisotropy screen described herein followed by performing the alpha screen described herein. In some aspects, the methods described herein can be carried out simultaneously or sequentially in any order.

In some aspects, the alpha screen can be the primary assay. In some aspects, the fluorescence polarization/anisotropy screen can serve as the primary assay. In some aspects, the alpha screen or the fluorescence polarization/anisotropy screen can serve as an orthogonal assay to the other screen/assay. In some aspects, the orthogonal assay can be performed following the primary assay. In some aspects, the orthogonal assay can be performed following the primary assay to differentiate between compounds that generate false positives from those compounds that are active against the target.

Kits

The kits described herein can include any combination of the compositions (e.g., one or more of the MSUT2 inhibitors) described above and suitable instructions (e.g., written and/or provided as audio-, visual-, or audiovisual material). In some aspects, the kit comprises a predetermined amount of a composition comprising any one compositions disclosed herein. The kit can further comprise one or more of the following: instructions, sterile fluid, syringes, a sterile container, delivery devices, and buffers or other control reagents.

### EXAMPLES

### Example 1: Alpha Screen Identifies MSUT2 Inhibitors for Tauopathy-Targeting Therapeutic Discovery

Materials and Methods. RNA. 5′ biotin-labeled and unlabeled poly(A) RNA were purchased (IDT, sequences 5′Biotin-AAAAAAAAAAAAAAA-3′ (SEQ ID NO: 1) and 5′-AAAAAAAAAAAAAAA-3′ (SEQ ID NO: 2)). Both biotin-labeled and unlabeled RNA were diluted to 10004 in RNAse/DNAse free Qiagen water and stored at −80° C., away from light. RNA was diluted to working concentration in Alpha Assay buffer just before screening.

Recombinant Protein. MSUT2 ZF and PABNP1 cDNA were cloned into the pGEX-6P1 vector (Pharmacia). MSUT2 ZF and PABPN1 encoding plasmids were transformed into BL21 (DE3) bacteria. 10 mL Terrific Broth (TB) starter cultures were grown overnight at 37° C. in a shaking incubator. The following morning, 1 L TB cultures were inoculated and grown at 37° C. with shaking to log phase and induced with 1 mM final concentration IPTG for 4 hours at 37° C. Following induction, DNA and RNA was degraded using benzonase nuclease or a cocktail of DNAse I and RNAse A. Affinity based gravity column purification was performed by binding GST-tagged MSUT2 or PABPN1 to sepharose-glutathione resin and subsequently eluting with 20 mM glutathione. Resulting eluate was buffer exchanged into PBS and stored at −80° C. Protein purity and yield were analyzed via Bradford assay and Coomassie-stained SDS-PAGE.

Chemical Library. The Spectrum Collection was purchased from MicroSource and contained a total of 2000 compounds in DMSO. For screening, 50 nL compound was transferred via pin tool to a final screening concentration of 10 μM. Dose response curves were generated from the library plates. Powder stock based retesting of hits that passed these screens were purchased from Sigma.

Alpha Screen Assay. Samples were set up in 384 well Perkin Elmer white opaque-bottom plates (PE06). A total reaction volume of 154 was plated in 384 format using a CyBio Well Vario (6 μL of donor beads (10 μg/mL), 3 μL biotinylated RNA (250 nM), 3 μL GST-MSUT2 protein (250 nM) per well). 50 nL of compounds were transferred to wells via CyBio Vario equipped with Pin Tool. This mixture was incubated at room temperature for 30 minutes away from light. Next 3 μL of acceptor beads (1.25 μg/mL) was added. Plates were incubated at room temperature away from light for 60 minutes and subsequently read on a Perkin Elmer EnVision multimode microplate reader equipped with stackers using a standard 384-well Alpha Assay software protocol.

Fluorescence Polarization Assay. Follow-up fluorescence polarization assay was performed in a ½ area black plate (Corning 3686). 504 of 125 nM MSUT2 and 10 nM FAM-RNA (ordered from IDT) in PBS were transferred using an Integra Viaflo with 96/50 uL head. 2 μL of compound was transferred for a final concentration of 10 μM. Plates were incubated for 20 minutes at room temperature and read using a Cytation 5 with pre-configured green polarization filter cube (8040561) at excitation 485/20 emission 528/20 and dichroic mirror at 510 nm and a read height of 10 mm. Fluorescence polarization was calculated by first subtracting background from a buffer-only control well and then using the equation

$$P = \frac{{F}_{∥} - {F}_{⊥}}{{F}_{∥} + {F}_{⊥}}$$

to determine polarization (P).

Cell culture and cytotoxicity screen. HEK293 cultured with cell growth medium: DMEM, 10% defined fetal bovine serum, Penicillin (1000 IU/mL) Streptomycin (1000 μg/mL) (Guthrie, C. R., et al., Hum Mol Genet 2011, 20 (10), 1989-99). Cell viability was assessed as using Promega Cell Titer Glo (Promega G7570) per manufacturer protocol. Briefly, HEK-293 cells were grown to 70% confluence in a 96 well plate were treated with indicated concentrations of compounds and incubated for 72 hours at 37° C. Plates were read for luminescence on Perkin Elmer Enspire Alpha.

Statistical analyses and figures. Graphs generated with GraphPad Prism 8. 4-parameter non-linear regression used to calculate indicated IC50 via GraphPad Prism 8.

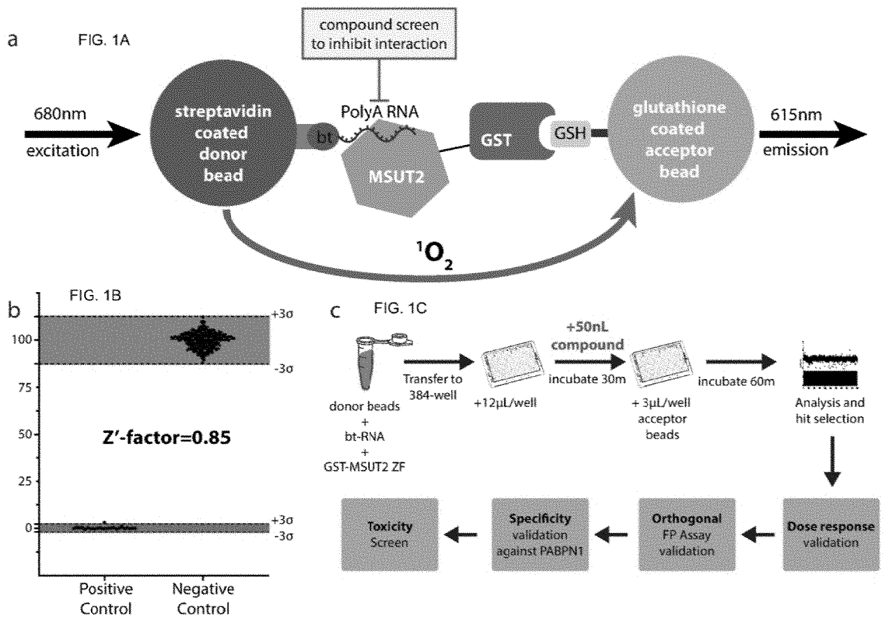

Results. Alpha Screen design and underlying principle. Alpha Screen technology relies on donor beads, coated with a hydrogel excitable by 680 nm, brought within close proximity to an acceptor bead leading to light emission and detection. In this assay, streptavidin coated donor beads tightly bind to biotinylated-poly(A) RNA while the glutathione-coated acceptor bead binds to GST-tagged MSUT2 protein. Because of the known high affinity of MSUT2 for poly(A) (K_{d}=60±15 nM){Wheeler, 2019 #58} beads are brought within the needed radius required for the donor bead to excite the acceptor bead via singlet oxygen transference. When this interaction is blocked by an inhibitor, the proximity threshold for donor and acceptor beads is not met, singlet oxygen transference cannot occur, and light is not emitted by the acceptor bead (FIG. 1a). Since at the outset, there were no known MSUT2 inhibitors, the positive control for inhibition in this assay was a high concentration of SDS which unfolds MSUT2 and effectively blocked bead to bead transference, while our negative control was the compound solvent, DMSO.

The calculated Z′-factor

$$( equation {Z}^{′} \text{-} factor = 1 - \frac{3 ( {σ}_{p} + {σ}_{n} )}{\vert {μ}_{p} - {μ}_{n} \vert} ,$$

σ=standard deviation, μ=mean, p=positive controls, n=negative controls) was 0.85 (FIG. 1b). This Z′-factor indicates our assay as robust {Zhang, 1999 #. The methodology used for tool compound validation first consisted of a primary Alpha Screen, where 50 nL of compounds were transferred via pin-tool to 384-well plates. After initial selection, hits were further validated by a broader range dose response, an orthogonal fluorescence polarization screen, for specificity, and finally for toxicity in a human cell model (FIG. 1c).

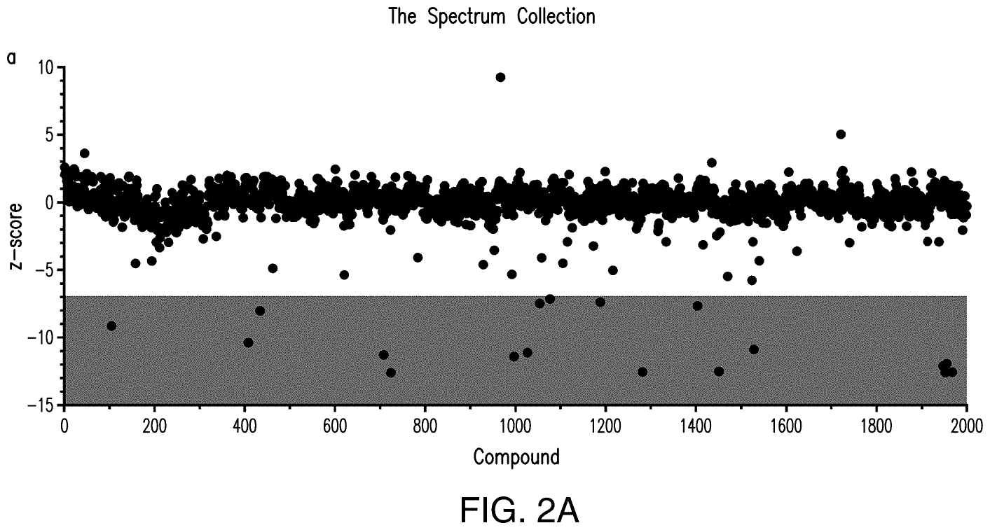

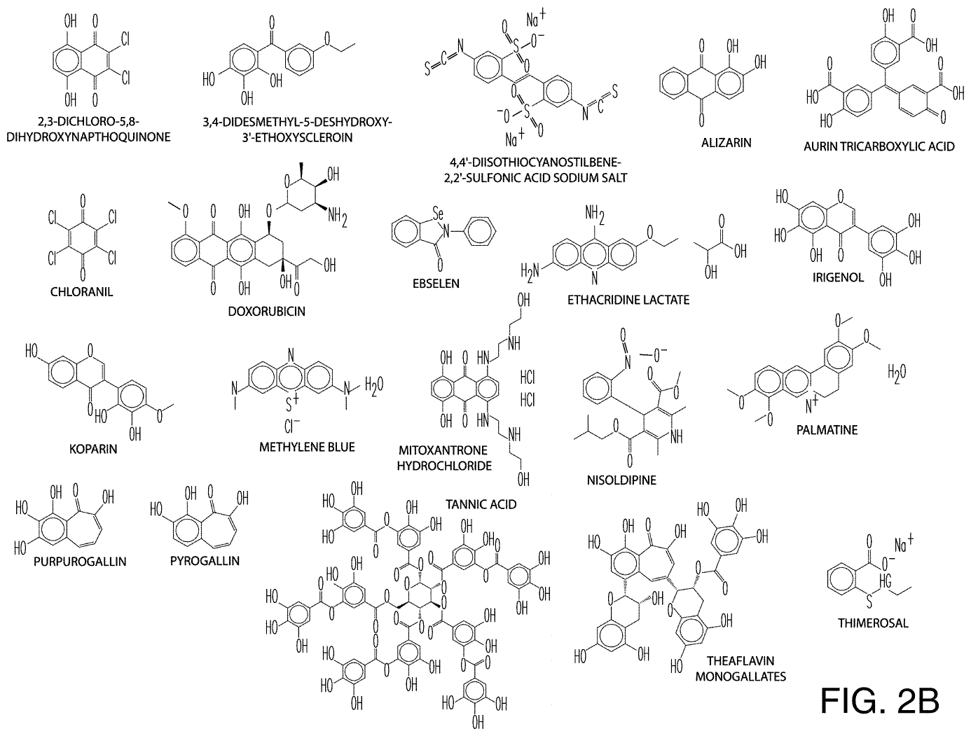

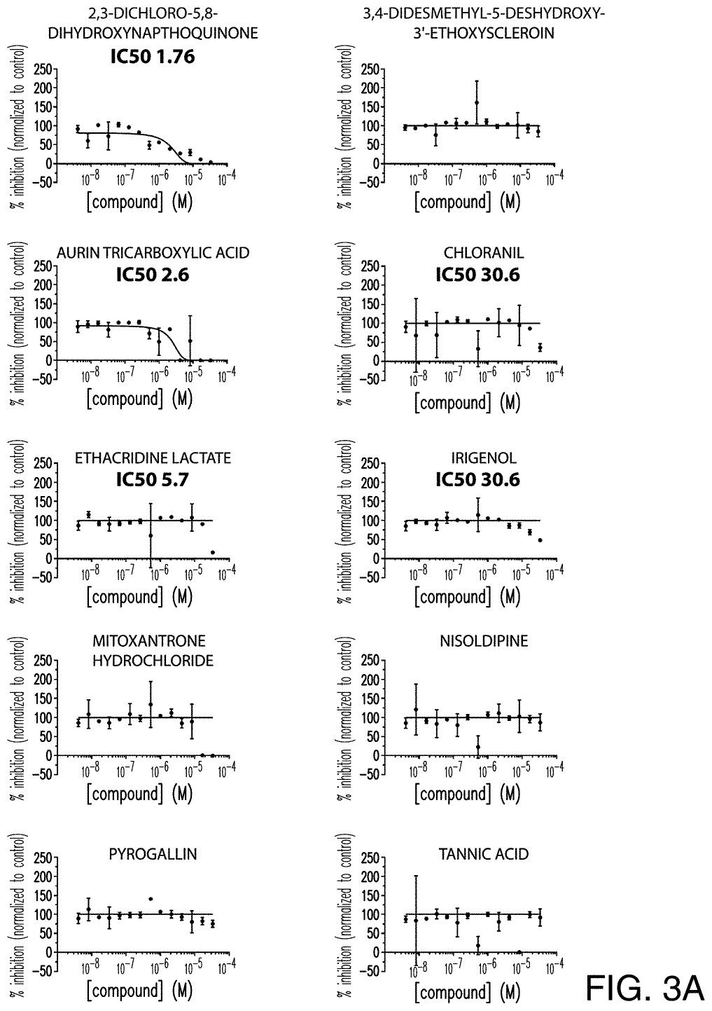

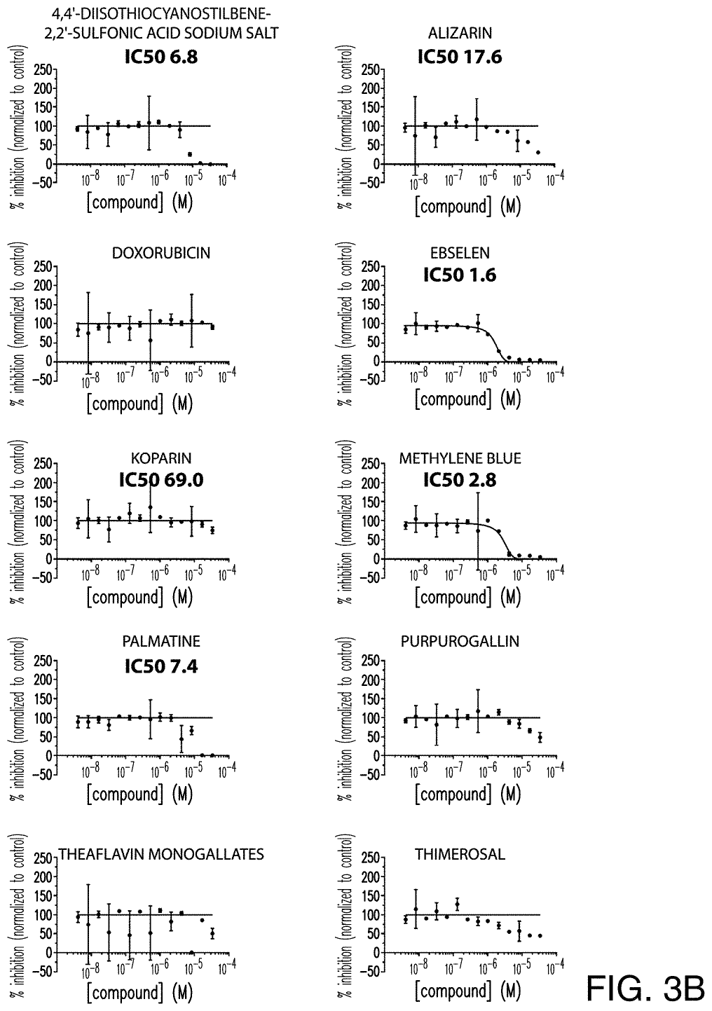

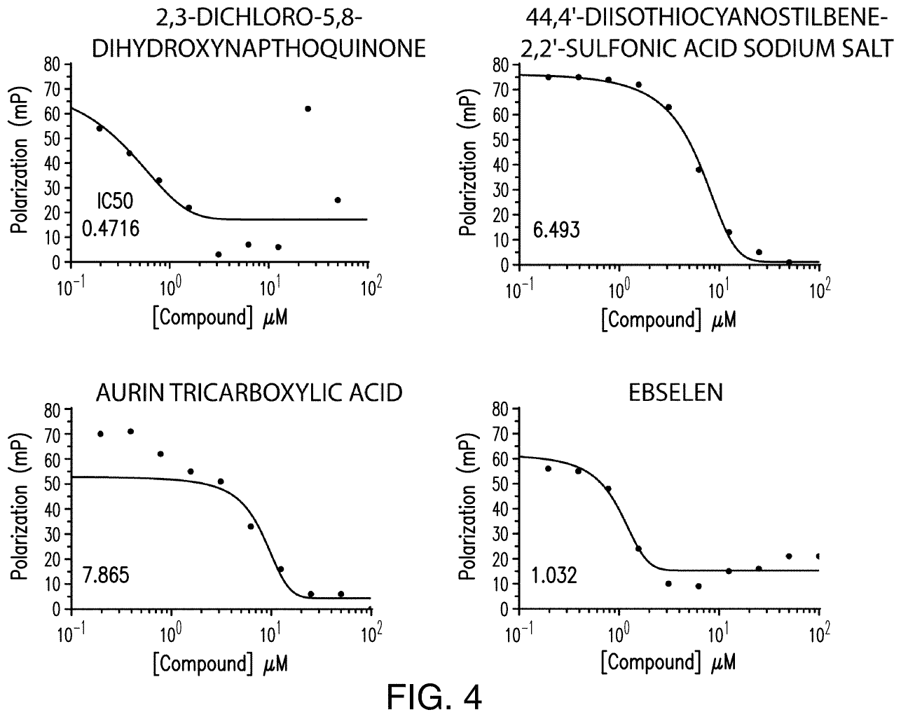

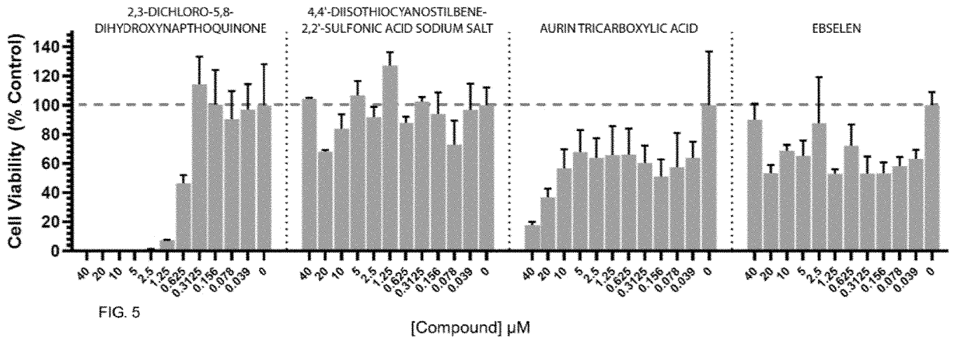

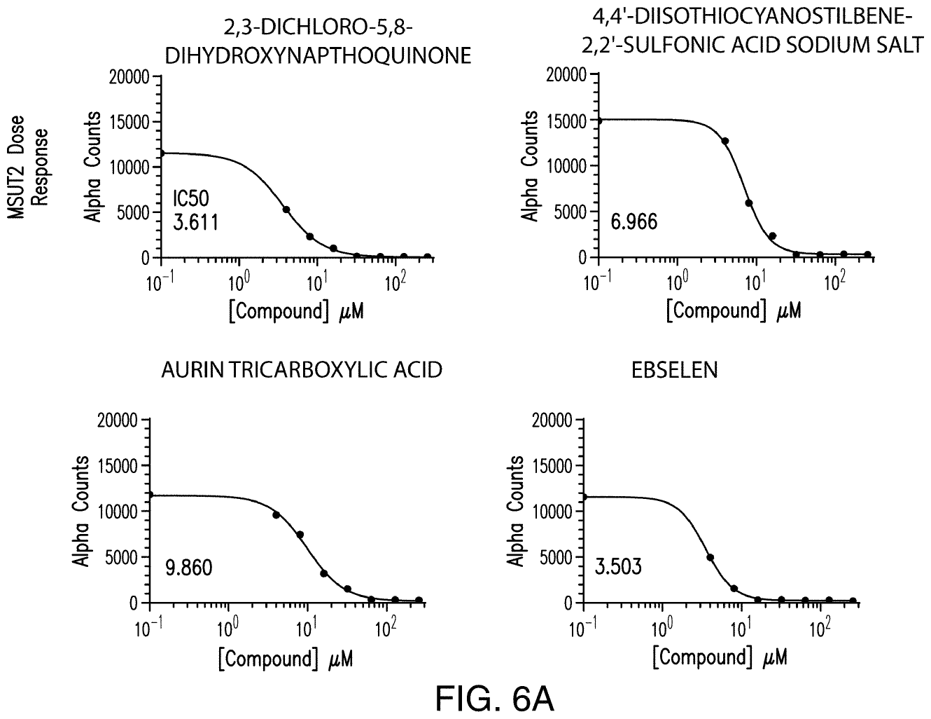

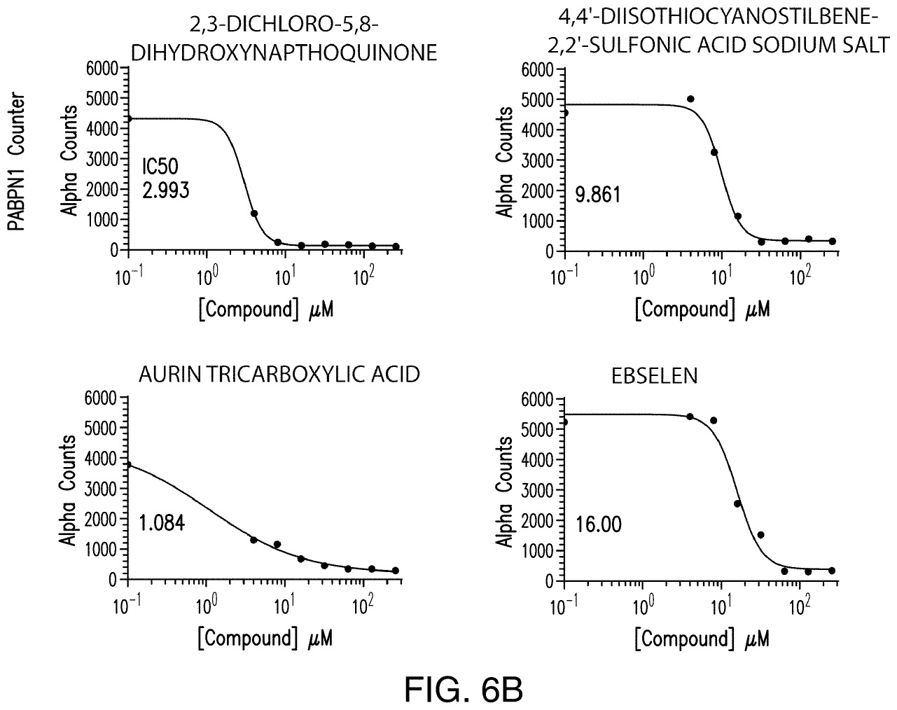

Screening of the Spectrum Collection. Screening of the 2000 compound Spectrum library resulted in the identification of 20 initial hits, for a hit rate of 2%. The hit window consisted of those compounds 7 standard deviations from the mean (FIG. 2a). Hit structures are indicated in FIG. 2b. In follow up dose-response testing, 11 of the 20 initial hits were validated and moved forward to further validation studies (FIG. 3). Unbound fluorescein-labeled RNA rapidly rotates in solution compared to MSUT2-bound. This results in a significant measurable reduction in polarized light emission, and is used to determine the small-molecule inhibition potential. This FP assay was used as an orthogonal assay to filter primary actives to 4 tool compounds: Ebselen, 2,3-dicholoro-5,8-dihydroxynapthoquinone, 4,4-diisothiocyanostilbene-2,2′-sulfonic acid, and Aurin tricarboxylic acid (FIG. 4). These four compounds were tested for an effect on cell viability using a HEK-cell model. While, 2,3-dicholoro-5,8-dihydroxynapthoquinone and Aurin tricarboxylic acid showed relatively high toxicity at high concentrations, 4,4-diisothiocyanostilbene-2,2′-sulfonic acid was relatively non-toxic at the doses tested. Ebselen did not show toxicity in a dose-dependent manner, but reduced cell viability by roughly 40% at the tested doses as compared to DMSO control treatment (FIG. 6). These four compounds were screened for specificity in an Alpha Screen against PABPN1, narrowing the field to Ebselen as a potent and selective in vitro inhibitor of the poly(A):RNA interaction (FIG. 5).

Discussion Tremendous unmet need exists for tau-modifying therapeutics to treat tauopathy disorders. Directly engaging tau shows promise as a strategy for treatment and there are a number of biologicals including tau antibodies and anti-tau vaccines as well as small-molecule aggregation inhibitors and PTM modifiers being tested for efficacy in patients with tauopathy disorders (primarily Alzheimer's disease and progressive supranuclear palsy) (Congdon, E. E., et al., Nature Reviews Neurology 2018, 14 (7), 399-415). Most small-molecule approaches directly targeting tau aggregation or phosphorylation have been abandoned for lack of efficacy or because of off-target complications and the majority of more advanced investigational therapeutics currently utilize immunotherapy based modalities (Cummings, J. L., et al., Alzheimers Res Ther 2014, 6 (4), 37-37). Some tau-targeting immunotherapies including, BMS-986168 (Gosuranemab), failed to show efficacy in PSP but are being investigated for AD-mediated mild cognitive impairment (ClinicalTrials.gov Identifier: NCT03068468), and Abbvie's AADvac1 and C2N-8E12 are also in early stage clinical trials (Congdon, E. E., et al., Nature Reviews Neurology 2018, 14 (7), 399-415). Determining which tau species to target has been a challenge because pathological tau encompasses a variety of misfolded or aberrantly modified monomeric tau species, toxic gain-of-function oligomers, paired helical filaments, as well as neurofibrillary tangle deposits (Brunden, K. R., et al., Nature Reviews Drug Discovery 2009, 8 (10), 783-793; Shafiei, S. S., et al., Front Aging Neurosci 2017, 9, 83-83; Ait-Bouziad, N., et al., Nature Communications 2017, 8 (1), 1678; Pooler, A. M., et al., Alzheimers Res Ther 2013, 5 (5), 49; and Dujardin, S., et al., Acta Neuropathologica Communications 2018, 6 (1), 132).

MSUT2 has emerged as an alternative for indirectly targeting pathological tau. It has been shown that knocking out MSUT2 in mice (PS19) overexpressing aggressively aggregating P301S mutated human tau prevents toxic oligomeric tau and NFT deposits while preserving neuronal health in the hippocampus and memory as evaluated by the Barnes maze paradigm (Wheeler, J. M., et al., Science Translational Medicine 2019, 11 (523), eaao6545). These mice develop normally and do not have clear defects as a result of the loss of MSUT2. While it is known that MSUT2 has a role in poly(A) tail length, its precise pathological mechanism of action remains unclear. Mislocalization of MSUT2 from the nucleus to the cytoplasm may provide a toxic gain-of-function activity allowing it to induce pathological tau formation, but this has not been shown directly. It has also been thought that MSUT2 may cause poly(A) RNA, a polyanion, to seed the pathological aggregation of tau. Regardless of the exact mechanism by which MSUT2 leads to tau accumulation, knocking down MSUT2 specifically effects toxic tau species including oligomers and NFTs (Guthrie, C. R., et al., Hum Mol Genet 2011, 20 (10), 1989-99; Wheeler, J. M., et al., Science Translational Medicine 2019, 11 (523), eaao6545; and Wheeler, J. M., et al., Biochem Soc Trans 2010, 38 (4), 973-6).

Traditionally, many RBPs and specifically the RNA:RBP interaction have been considered less than ideal for small molecule screening campaigns as there isn't a targetable enzymatic pocket. However, the results described herein have shown with two screening paradigms the ability to identify compounds that are specific and potent in blocking the poly(A):MSUT2 interaction. With the application of Alpha Screen and Fluorescence polarization technology, high-throughput screening initiatives for RNA-protein inhibitors have been successful for various RBP targets (D'Agostino, V. G., et al., PLoS One 2013, 8 (8), e72426; Jazurek, M., et al., Nucleic Acids Res 2016, 44 (19), 9050-9070; and Mills, N. L., et al., J Biomol Screen 2007, 12 (7), 946-55). The compound identified here, Ebselen, was shown to be potent for MSUT2 inhibition and had a 5-fold reduction in potency against PABPN1, the counter screen target. Because MSUT2 neuronal abundance is dwarfed by PABPN1, this level of specificity may be sufficient, however, it is possible that a higher specificity will be required for translationally effective compounds.

Ebselen has been investigated for broad clinical usage from general anti-inflammatory and antioxidant properties to specific uses in treatment of stroke, neurodegeneration, and for bipolar disorder (Schewe, T., General Pharmacology: The Vascular System 1995, 26 (6), 1153-1169; Yamaguchi, T., et al., Stroke 1998, 29 (1), 12-17; Singh, N., et al., Nature communications 2013, 4, 1332-1332; and Slusarczyk, W., et al., Neural Regen Res 2019, 14 (7), 1255-1261). There have been previous reports identifying neuroprotective effects in rodent models of both stroke (Takasago, T., et al., Br J Pharmacol 1997, 122 (6), 1251-1256) and Alzheimer's disease (Xie, Y., et al., JBIC Journal of Biological Inorganic Chemistry 2017, 22 (6), 851-865). In mice expressing three AD-associated mutations (Tau P301L, APP KM670/671NL, and PSEN1 M146V), oxidative stress, levels of amyloid-β, and hyper phosphorylated tau were reduced after Ebselen treatment via drinking water (Xie, Y., et al., JBIC Journal of Biological Inorganic Chemistry 2017, 22 (6), 851-865). The precise neuroprotective mechanism is unknown, however it is thought that the selenium containing Ebselen primarily works through prevention of oxidative damage (Xie, Y., et al., JBIC Journal of Biological Inorganic Chemistry 2017, 22 (6), 851-865).

Because of recent success in gene and transcript-targeting therapeutics, alternative approaches to target MSUT2 are considered. Antisense oligonucleotides (ASOs), single stranded DNA molecules that modulate mRNA have shown great success in the treatment of spinal muscular atrophy (SMA) and may serve as a therapeutic option in disrupting MSUT2 expression (Wurster, C. D., et al., Ther Adv Neurol Disord 2018, 11, 1756285618754459-1756285618754459). An alternative to ASOs, siRNAs work to reduce expression through the recruitment of RNA-induced silencing complex (RISC) and subsequent message degradation. siRNAs may be more efficacious than ASOs in regards to MSUT2-targeting, as it is thought that MSUT2 must be strongly deactivated for therapeutic effect as heterozygous MSUT2 knockout mice are not protected from tau-mediated neurodegeneration (Watts, J. K., et al., J Pathol 2012, 226 (2), 365-379). Because of the relatively short half-life of oligonucleotides and their inability to cross the blood-brain barrier, administration is problematic as it requires multiple, life-long, intrathecal injections (Pattali, R., et al., Gene Therapy 2019, 26 (7), 287-295). Because of these concerns, gene-targeting strategies may be preferred and have been proven efficacious. Onasemnogene abeparvovec, a gene therapy for treatment of SMA, delivers a functional SMN1 gene via an AAV9 vector with a single intravenous dose (Pattali, R., et al., Gene Therapy 2019, 26 (7), 287-295).

MSUT2 is a promising target in combatting tau mediated neurodegeneration. Advancing the understanding of fundamental MSUT2 mediated mechanisms of neurodegeneration remains an important component of therapeutic development. The identification of these initial inhibitors will allow MSUT2 function to be probed and to facilitate the development of more translationally suitable MSUT2 inhibitors. Likewise, solving the structure and determining the pathological mechanism of MSUT2 will present new opportunities in targeting MSUT2-induced tau pathology. A solved structure will allow for virtual docking of compounds and more precise medicinal chemistry initiatives for developing small molecules, while determining its role in biological and pathological pathways will allow for reducing off-target complications and may provide new therapeutic targets.

### Example 2: Targeting Pathological Tau by Small Molecule Inhibition of the Poly(A):MSUT2 RNA-Protein Interaction

Neurofibrillary tangles composed of aberrantly aggregating tau protein are a hallmark of Alzheimer's disease and related dementia disorders. Recent work has shown mammalian suppressor of tauopathy 2 (MSUT2) controls accumulation of pathological tau in cultured human cells and mice. Knocking out MSUT2 protects neurons from neurodegenerative tauopathy and preserves learning and memory. MSUT2 protein functions to bind poly adenosine [poly(A)] tails of messenger RNA through its C-terminal CCCH type zinc finger domains and loss of CCCH domain function suppresses tauopathy in C. elegans and mice. Thus, it was tested whether inhibiting the poly(A):MSUT2 RNA-protein interaction would ameliorate pathological tau accumulation. Described herein is a high-throughput screening method for the identification of small molecules inhibiting the poly(A):MSUT2 RNA-protein interaction. A fluorescent polarization assay was used for initial small molecule discovery with the intention to repurpose hits identified from the NIH Clinical Collection (NIHCC). The drug repurposing development workflow included validation of hits by dose response analysis, specificity testing, orthogonal assays of activity, and cytotoxicity. Validated compounds passing through this screening funnel will be evaluated for translational effectiveness in future studies. This pre-clinical drug development pipeline identified diverse FDA approved drugs Duloxetine, Saquinavir, and Clofazimine as potential repurposing candidates for reducing pathological tau accumulation.

Introduction. Alzheimer's disease (AD) causes progressive impairment of cognitive function due to neurodegeneration and atrophy in the brain regions responsible for learning and memory; there are no known effective disease modifying therapies (Congdon, E. E. & Sigurdsson, E. M. Nature Reviews Neurology 14, 399-415, (2018)). As the population of the United States has aged, the prevalence of AD has increased (Corrada, M. M., et al., Ann Neurol 67, 114-121, (2010)). To put the problem in perspective, heart disease-related deaths dropped 9% between 2000 and 2017, while AD-related deaths increased by 145% (2019 Alzheimer's disease facts and figures. Alzheimer's & Dementia 15, 321-387, (2019)). AD histopathology consists of neurofibrillary tangles (NFTs) composed of aberrantly aggregating tau protein within neurons and senile plaques composed of amyloid-beta (AP) in the interneuronal space (Wood, J. G., et al., Proc Natl Acad Sci USA 83, 4040-4043, (1986); Glenner, G. G. & Wong, C. W. Biochem Biophys Res Commun 425, 534-539, (2012); and Bloom, G. S. JAMA Neurol 71, 505-508, (2014)). The complex molecular dynamics underlying AD pathology remain incompletely understood and the precise cause of disease initiation and progression remain unclear for late onset Alzheimer's disease (Hanseeuw, B. J. et al. JAMA Neurology 76, 915-924, (2019)). However, pathological tau burden detected post-mortem by conventional brain histology or ante-mortem using PET imaging tools show pathological tau burden correlates well with cognitive decline (Arriagada, P. V., et al., Neurology 42, 631-639, (1992); and Xia, C. et al. JAMA Neurol 74, 427-436, (2017)). Although a hallmark of AD, NFTs also appear in many other distinct dementia disorders. Disorders exhibiting deposits of pathological tau protein are known as tauopathy disorders and include frontotemporal lobar degeneration, Pick's disease, progressive supranuclear palsy, corticobasal degeneration, and AD (Rojas, J. C. & Boxer, A. L. Nature Reviews Neurology 12, 74-76, (2016)). Therapeutics targeting tau may have broad impact across these tauopathy disorders.

Although extensive drug-discovery initiatives primarily targeting the pathologically aggregating peptide AP have been ongoing for over two decades, this approach has failed to yield viable therapeutics to halt neurodegeneration (Huang, Y. & Mucke, L. Cell 148, 1204-1222, (2012)). Recent work has suggested that RNA binding proteins (RBPs) may play an important role in the progression of diverse neurodegenerative diseases by interacting with the messenger RNA (mRNA) of aberrantly aggregating proteins (Wolozin, B. & Ivanov, P. Nature Reviews Neuroscience 20, 649-666, (2019)). These studies suggest targeting RBPs could have future therapeutic utility.

The RBP family of proteins is diverse and is comprised of over 1,500 genes in humans (Gerstberger, S., et al., Nature Reviews Genetics 15, 829-845, (2014)). The canonical functions of RBPs reflect this diversity and range from RNA transcription, splicing, polyadenylation, RNA export, localization, and translation (Hentze, M. W., et al., Nature Reviews Molecular Cell Biology 19, 327-341, (2018)). TAR DNA-binding protein 43 (TDP-43) has a well-defined role in neurodegeneration (Arai, T. et al. Biochemical and Biophysical Research Communications 351, 602-611, (2006)). TDP-43, expressed in the nucleus, functions in RNA transport and processing (Ito, D., et al., Science Translational Medicine 9, eaah5436, (2017)). The accumulation and mislocalization of mutated TDP-43 into insoluble cytoplasmic deposits occurs in the majority cases of Amyotrophic Lateral Sclerosis (Neumann, M. et al. Science 314, 130-133, (2006)). Other RBPs implicated in neurodegenerative disorders include FUS in ALS, as well as a repeat expansion in PABPN1 causing oculopharyngeal muscular dystrophy (Brais, B. et al. Nat Genet 18, 164-167, (1998); Kwiatkowski, T. J., Jr. et al. Science 323, 1205-1208, (2009); and Vance, C. et al. Science 323, 1208-1211, (2009)).

Previous work has shown the poly(A)-binding protein MSUT2 (known also as ZC3H14) potentiates pathological tau accumulation and may serve as a tractable therapeutic target for intervention in tauopathies including Alzheimer's disease (Guthrie, C. R., et al., Hum Mol Genet 20, 1989-1999, (2011); and Wheeler, J. M. et al. Science Translational Medicine (2019)). It has been shown that RNAi knockdown of MSUT2 in a human cell model overexpressing tau decreases pathological tau species including phosphorylated tau, pre-tangle conformations and detergent insoluble tau species. Further and most compelling, tau transgenic MSUT2 knockout mice are protected against tau-mediated neurofibrillary degeneration including decreased pathological tau burden, reduced memory deficits, and neuronal preservation (Wheeler, J. M. et al. Science Translational Medicine (2019)).

An important consideration in drug discovery for inhibitors of the poly(A):MSUT2 RNA-protein interaction is specificity. MSUT2 works in concert with another important regulator of RNA processing, Poly(A) Binding Protein Nuclear 1 (PABPN1) (Wigington, C. P., et al., Wiley Interdiscip Rev RNA 5, 601-622, (2014)). PABPN1 is expressed in the nucleus throughout tissues and binds to RNA to control the size of mRNA transcript poly(A) tails (Brais, B. et al. Nat Genet 18, 164-167, (1998)); and Wahle, E. Cell 66, 759-768, (1991)). It has been shown that MSUT2, PABPN1, and poly(A) RNA colocalize in nuclear speckles (Guthrie, C. R., et al., Hum Mol Genet 20, 1989-1999, (2011)). Any therapeutic strategy targeting MSUT2 binding to poly(A) RNA must avoid altering the poly(A):PABPN1 interaction because PABPN1 knock down exacerbates pathological tau accumulation in cultured cells (Wheeler, J. M. et al. The poly(A) binding protein MSUT2 controls resistance to both pathological tau and gliosis. Science Translational Medicine (2019)) and PABPN1 serves as an essential protein (knockout lethal) (Malerba, A. et al. Nature Communications 8, 14848, (2017)).

Described herein are methods using a drug repurposing pipeline for the identification of potent and specific small-molecule inhibitors of MSUT2 RBP and poly(A) RNA. To this end, a primary fluorescence polarization (FP) high-throughput screen (HTS) identified compounds which were further validated through a PABPN1 counter-screen, an orthogonal Alpha Screen, and for cell toxicity.

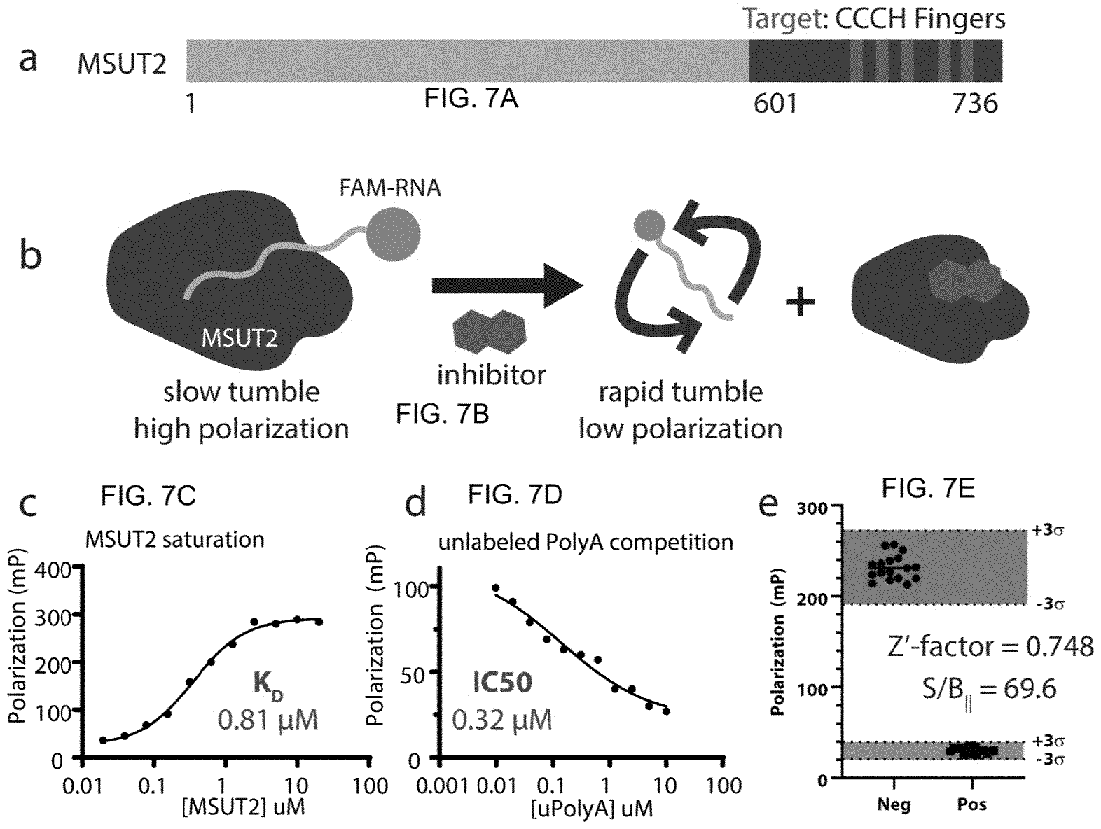

Results and Discussion. A high-throughput fluorescence polarization assay for inhibition of poly(A):MSUT2 RNA interaction. Fluorescence polarization relies on the fact that apparent rotational velocity (tumbling) of molecules is inversely proportional to molecular weight (Jameson, D. M. & Ross, J. A. Chem Rev 110, 2685-2708, (2010)). Polarized incident light striking a relatively large, slowly tumbling fluorescently tagged molecule is emitted as polarized light while small, rapidly tumbling molecules emit non-polarized light (Weber, G. Biochem J 51, 145-155, (1952)). In order to screen potential inhibitors of MSUT2 RBP and RNA by FP, fluorescent FAM (fluorescein amidite)-labeled poly(A)₁₅ RNA (FAM-RNA) and recombinant MSUT2 ZF (zinc finger) protein expressing constructs were generated (FIG. 7a). Recombinant MSUT2 ZF protein and FAM-RNA form a complex (FAM-RNA:MSUT2) in vitro with high affinity. This complex tumbles at a relatively low rate in comparison to free FAM-RNA resulting in a high emission of polarized light (FIG. 7b). Inhibition of the interaction results in unbound FAM-RNA tumbling at a relatively rapid rate and emitting non-polarized light (FIG. 7b).

To determine the concentrations of both MSUT2 ZF protein and FAM-RNA probe useful in the methods described herein, a two dimensional titration of protein and RNA concentrations in 96-well format was performed. This experiment revealed that a ratio of 10 nM FAM-RNA:125 nM MSUT2 ZF resulted in robust and reproducible polarization signal along with a sufficiently high fluorescence intensity to minimize any potential background interference. Next, the binding constant between MSUT2 ZF and FAM-RNA was determined by holding FAM-RNA constant at 10 nM while increasing MSUT2 ZF concentration. An affinity of 0.81 μM for FAM-RNA:MSUT2 ZF interaction was determined (FIG. 7c). To develop a positive control for inhibition of FAM-RNA:MSUT2 ZF complex formation, d varying concentrations of unlabeled poly(A)₁₅ RNA in an FP competition assay (10 nM FAM-RNA:125 nM MSUT2 ZF) was tested resulting in an IC50 of 0.32 μM (FIG. 7d).

Z′-factor is a well-known statistic for suitability of high-throughput screening (HTS) design (Zhang, J. H., et al., J Biomol Screen 4, 67-73, (1999)). The Z′-factor of our screen, 0.748, was calculated using the equation

$${Z}^{′} \text{-} factor = 1 - \frac{3 ( {σ}_{p} + {σ}_{n} )}{\vert {μ}_{p} - {μ}_{n} \vert} ,$$

where σ=standard deviation, μ=mean, p=positive controls, and n=negative controls (FIG. 7e). This score indicates a robust assay suitable for screening and identification of potential FAM-RNA: MSUT2 ZF interaction inhibitors. Additionally, experimental conditions provided for a high signal to background ratio of 69.6 (FIG. 7e).

A drug repurposing strategy: screening of the NIH Clinical Collection compound library. Because of the rapidly increasing costs of drug development from initial screening through clinical trials, repurposing approved drugs for new indications has become an important focus in drug development (Pushpakom, S. et al. Nature Reviews Drug Discovery 18, 41, (2018)). The plan to find suitable lead compounds for drug repurposing began with a screen of the NIH Clinical Collection (NIHCC) library. The NIHCC consists of a diverse array of 700 compounds with historical pharmacological use and well-studied safety profiles. The workflow for identifying MSUT2 selective inhibitors included a primary FP screen followed by dose validation. An orthogonal poly(A):MSUT2 binding assay allowed the ruling out of assay interference while selectivity against MSUT2 over PABPN1 was determined by a parallel FP assay with PABPN1 as the RBP. PABPN1 knockdown strongly exacerbates tau accumulation in human cells while Alzheimer's cases exhibiting depletion of the MSUT2/PABPN1 complex show more severe neurodegenerative changes. Thus, the goal was to eliminate compounds interfering with both PABN1 and MSUT2 and focus on compounds truly specific for poly(A):MSUT2 binding. From the potent, dose responsive, and selective compounds inhibiting MSUT2 RNA binding, hits were validated for low toxicity and considered suitable for further studies in physiological relevant models.

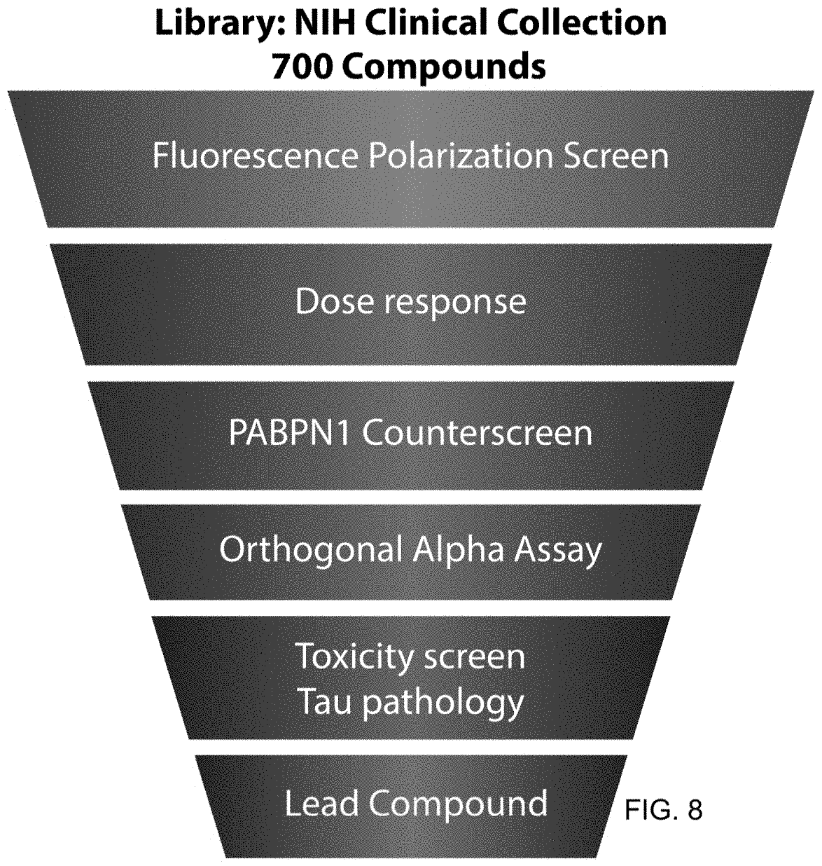

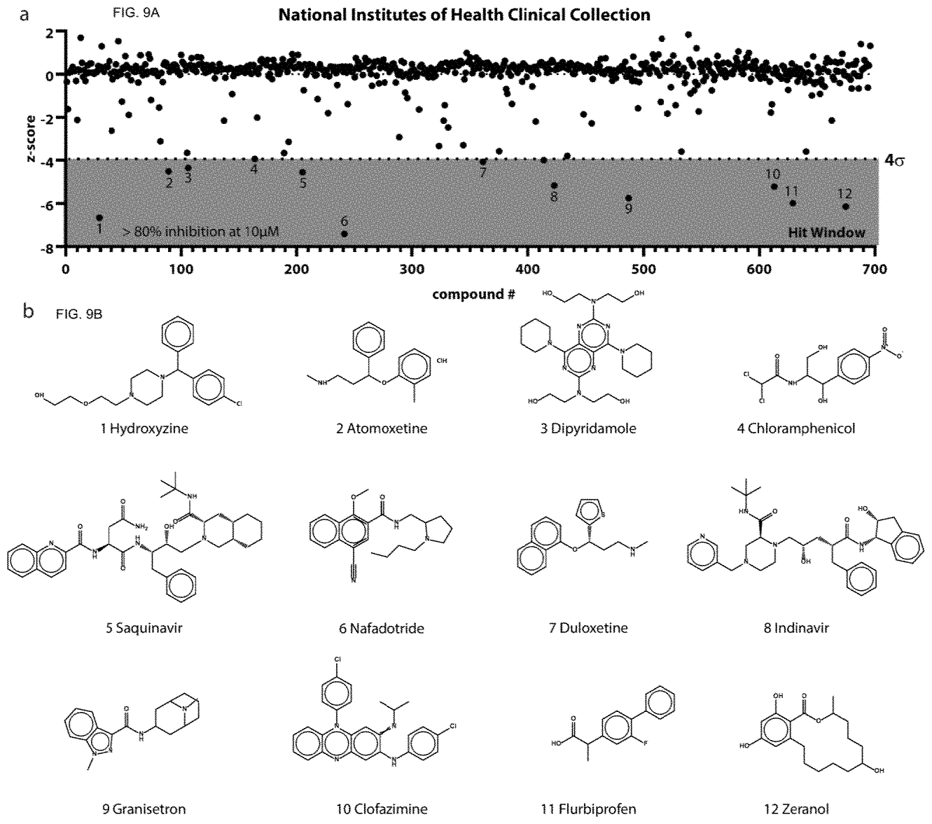

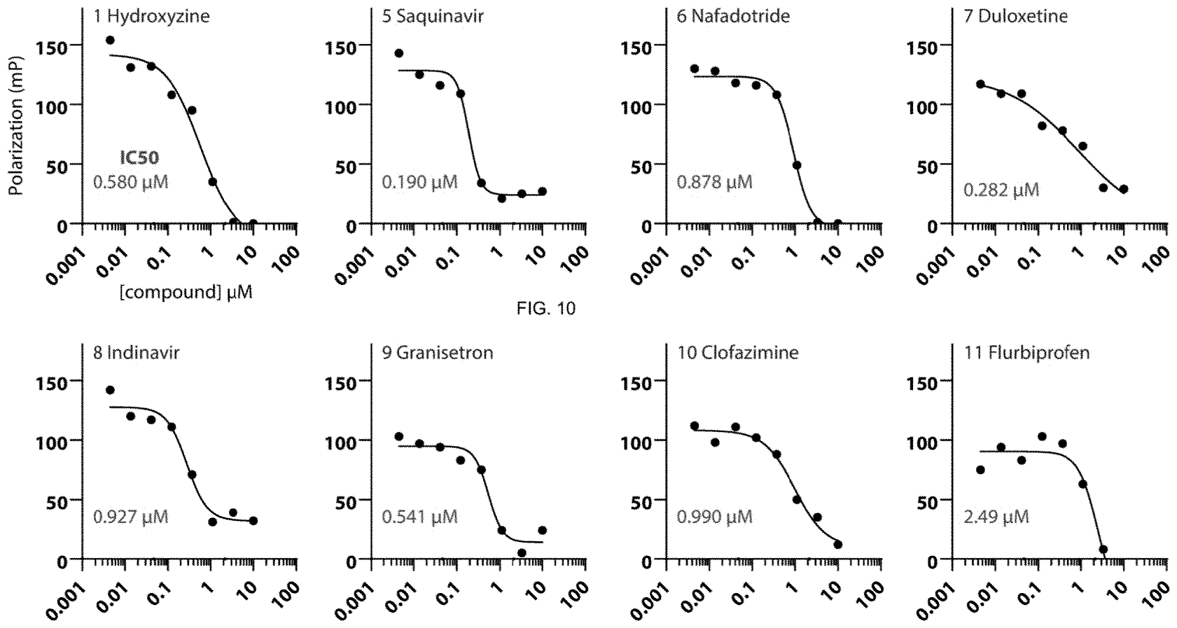

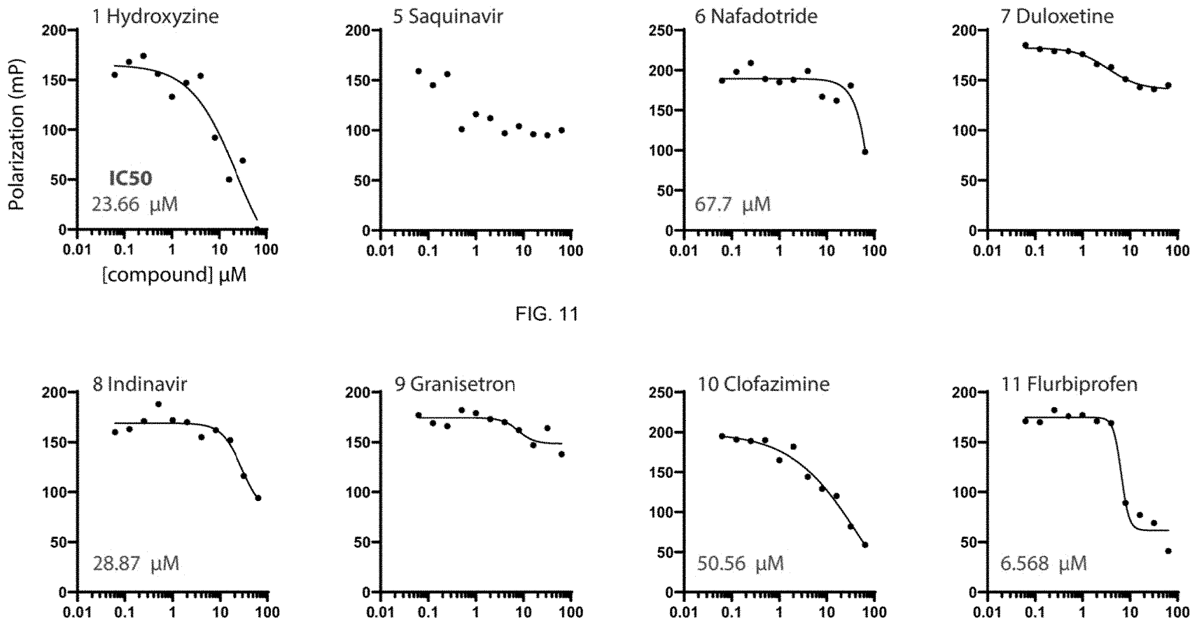

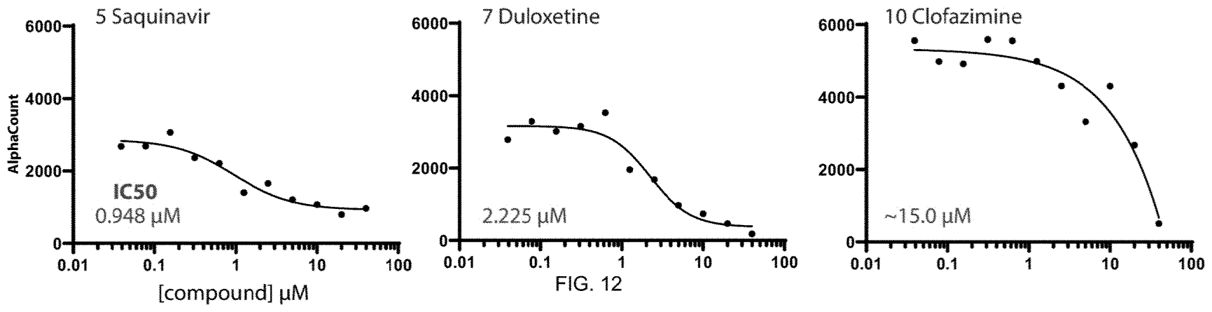

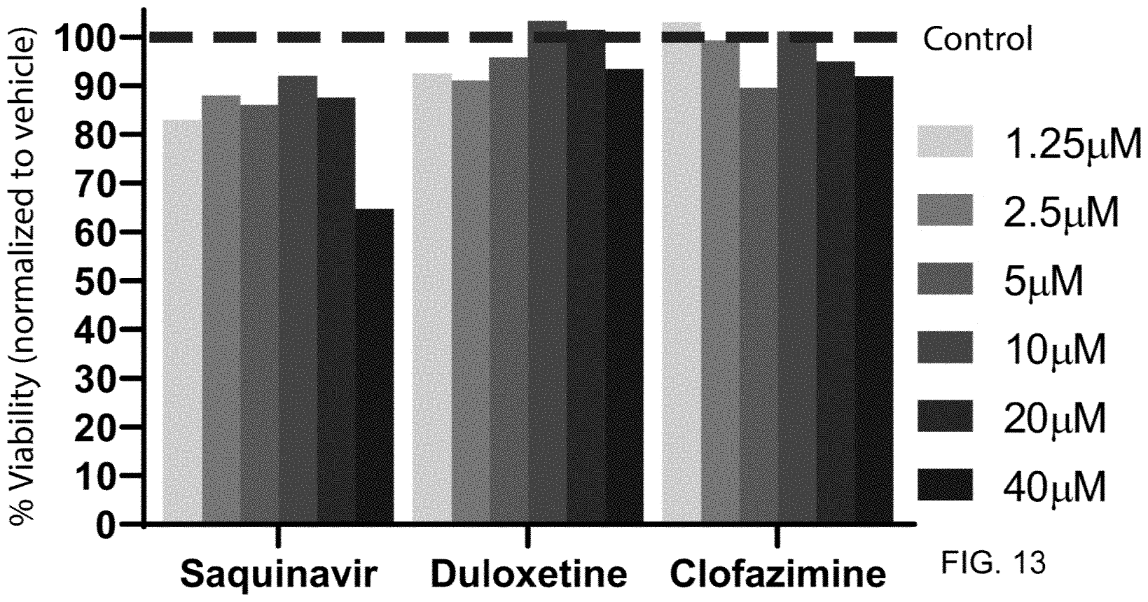

Identification and validation of MSUT2 inhibitory compounds from NIHCC library. The single-point primary screen was conducted in duplicate and utilized the FP assay conditions determined herein (e.g., 125 nM MSUT2 RNABP and 10 nM FAM-RNA probe) with 10 μM library compounds added in a 96-well format. This screen resulted in 12 initial hits (FIG. 9a). The hit window corresponded to those compounds with >80% inhibition at 10 μM and corresponded to Z-scores ≤−4σ (standard deviations) from the mean polarization of the samples (FIGS. 9a&b). Hits were further dose-response validated by fluorescence polarization, and 8 compounds showed dose-dependent inhibition (FIG. 10). The hits were relatively potent, with IC50s below 3 μM (FIG. 10). Compound specificity was empirically tested by counter screening against PABPN1, a regulator of the length of mRNA poly(A) tails (Apponi, L. H. et al. Hum Mol Genet 19, 1058-1065, (2010); and Benoit, B. et al. Dev Cell 9, 511-522, (2005)). IC50 of each of the previous 8 compounds was determined for PABPN1 using fluorescence polarization (FIG. 11), and a specificity ratio determined (Table 3). An Alpha Screen (AS) assay was developed as an orthogonal screen. Briefly, in this screen, biotinylated RNA binds streptavidin-coated donor beads, while GST-MSUT2 ZF binds glutathione-coated acceptor beads. When donor beads and acceptor beads are brought within close proximity of one another, laser excitation leads to fluorophore emission of the acceptor bead via excited singlet oxygen activating chemiluminescers in the acceptor beads. Active compounds prevent beads from being arranged in close proximity, and decrease Alpha signal. Compounds were considered validated if they showed activity both by FP and AS. Duloxetine, Saquinavir, and Clofazimine displayed dose-dependent inhibition activity by AS (FIG. 11) and were further tested for toxicity. Toxicity of these compounds in a HEK cell model was determined using a standard Promega Cell Titer Glo assay. Saquinavir showed some toxicity at the highest tested dose of 4004 (65% viability), while Duloxetine and Clofazimine viability was above 90% for all tested doses when normalized to 2% DMSO vehicle control (FIG. 10a).

TABLE 3
IC50 of select compounds determined for PABPN1 using
fluorescence polarization and a specificity ratio.

| | IC50_{MSUT2} | IC50_{PABPN1} | PABPN1/MSUT2 |
|---|---|---|---|
| Hydroxyzine | 0.580 | 23.66 | 40.7931 |
| Saquinavir | 0.190 | None | NA |
| Nafadotride | 0.878 | 67.7 | 77.10706 |
| Duloxetine | 0.282 | None | NA |
| Indinavir | 0.927 | 28.87 | 31.14347 |
| Granisetron | 0.541 | None | NA |
| Clofazimine | 0.990 | 50.56 | 51.07071 |
| Flurbiprofen | 2.490 | 6.568 | 2.637751 |

The Utility of Drug Repurposing. The NIH Clinical Collection (NIHCC) library was screened for small molecules that inhibit the poly(A):MSUT2 RNA-protein interaction as a first effort at MSUT2 drug development. The NIHCC is suited for drug repurposing, a drug development approach that seeks to discover new indications for previously approved drugs (Sleigh, S. & Barton, C. Pharmaceutical Medicine 24, 151-159, (2010); and Pushpakom, S. et al. Nature Reviews Drug Discovery 18, 41, (2018)). The advantages realized by drug repurposing include the elimination of over a decade's worth of preclinical drug discovery work and streamlined clinical trials. This approach has become particularly important when one considers that drug development spending has doubled while very few therapeutics targeting neurodegeneration have proven efficacious (Cummings, J. L., et al., Alzheimers Res Ther 6, 37-37, (2014)). Drug collections suitable for repurposing include safe drugs which may have failed to show efficacy in clinical trials, off-patent generics, or those abandoned because of commercial reasons (Sleigh, S. & Barton, C. Pharmaceutical Medicine 24, 151-159, (2010)). Efforts to find novel indications for existing drugs is largely driven by the extensive costs in time and money to bring drugs to market, estimated at 13 years and $1.8 billion on average (Gupta, S. C., et al., Trends in Pharmacological Sciences 34, 508-517, (2013)). Highlighting the merit to this approach, 90% of blockbuster drugs from 1993 now have secondary indications (Gelijns, A. C., et al., New England Journal of Medicine 339, 693-698, (1998)). Because safety and efficacy have already been well-established, drug candidates from repurposing collections such as the NIHCC have a much higher likelihood of reaching Phase II trials and beyond.

Therapeutic potential of MSUT2 as a target. Due to its recent identification as a driver of mammalian tauopathy, targeting MSUT2 for therapeutic development remains in the early preclinical discovery stages (Wheeler, J. M. et al. Science Translational Medicine (2019)). Targeting MSUT2 is challenging as MSUT2 is non-enzymatic and has no known targetable binding pocket. Additionally, while the structure of Nab2, the yeast homolog of MSUT2, has been determined by crystallography and NMR (Brockmann, C. et al. Structure 20, 1007-1018, (2012); and Kuhlmann, S. I., et al., Nucleic Acids Res 42, 672-680, (2014), the MSUT2 protein structure remains unsolved preventing extensive in silico drug discovery efforts. Furthermore, until the development of methods presented herein, there have been no adequate high-throughput screening (HTS) assays for MSUT2 function and inhibition. Moreover, bulk poly(A) RNA maintenance is a ubiquitous function and off-target effects are an important consideration. Despite these concerns MSUT2 does present an intriguing target for drug discovery given its strong effects on pathological tau and that MSUT2 function is dispensable for mouse development as MSUT2 knockout mice appear normal.

Overexpression of MSUT2 in tau-transgenic mouse hippocampi leads to increases in pathological tau deposition, neuroinflammation, and neurodegeneration. Conversely, knocking out MSUT2 in mouse models of tauopathy has the reciprocal effect and reduces hyper-phosphorylated, pre-tangle and tau tangle burden, while being anti-inflammatory and neuroprotective (Wheeler, J. M. et al. Science Translational Medicine (2019)). Further, MSUT2 knockout protects against memory deficits as shown by Barnes maze paradigm. MSUT2 knockout mice (in a non-tau background) are healthy, lack cognitive deficits, and display normal neurological function (Wheeler, J. M. et al. Science Translational Medicine (2019)). As mentioned previously, MSUT2 interacts with PABPN1 and forms a complex within neurons. Evidence suggests that while some MSUT2 positive neurons have tau tangles, depletion of the MSUT2:PABPN1 complex exacerbates Alzheimer's disease (AD) pathology with higher pathological tau burden, increased neuronal loss, and an early onset of AD (Wheeler, J. M. et al. Science Translational Medicine (2019)). Thus, PABPN1 would appear to be an important anti-target for poly(A):MSUT2 inhibitors.

Perspective on hit compounds and future screening. The screen methodology presented here identifies three potential repurposing candidates. Duloxetine, a serotonin-norepinephrine (SNRI) reuptake inhibitor known as Cymbalta, has indications for depression, anxiety, pain caused by neuropathy as well as fibromyalgia. Notably, while first approved for major depressive disorder in 2004, additional indications were not approved until years later: fibromyalgia in 2008 (Wright, C. L., et al., Expert Rev Clin Immunol 6, 745-756, (2010); and Lunn, M. P., et al., Cochrane Database Syst Rev, Cd007115, (2014)) and musculoskeletal pain in 2010 (Smith, H. S., et al., Ther Clin Risk Manag 8, 267-277, (2012)). Duloxetine became available as generic in 2013. Notably, a 10 year study of 20,215 elderly patients prescribed various serotonin reuptake inhibitors showed that duloxetine use was associated with reduced risk of dementia (Kostev, K., et al., J Alzheimers Dis 69, 577-583, (2019)). Further, in a rat model of chronic cerebral hypoperfusion, Duloxetine attenuated neuronal loss in the hippocampus (Park, J. A. & Lee, C. H. Biomol Ther (Seoul) 26, 115-120, (2018)). The mechanism for potential protection from dementia is unknown, and planned studies will look at the effects of Duloxetine in a mouse model of tauopathy to determine if it is suitable for further pre-clinical studies.

Saquinavir, developed by Roche under brand name Invirase®, is a protease inhibitor prescribed as an antiretroviral to treat HIV infection. Saquinavir mechanism of action is to bind and inhibit viral proteases HIV-1 and HIV-2 leading to the prevention of viral maturation (Pribis, J. P. et al. Mol Med 21, 749-757, (2015)). There is currently no secondary indications for Saquinavir, although there have been studies highlighting its activity against other targets. For example, a recent repurposing effort identified Saquinavir (as well as Clofazimine) as potential therapeutics for Chagas disease (Bellera, C. L. et al. European Journal of Medicinal Chemistry 93, 338-348, (2015)).

Clofazimine, though primarily used as a treatment for leprosy caused by Mycobacterium leprae, has been studied for other indications. Clofazimine is currently being looked at for its effectiveness in treating tuberculosis, caused by the related bacteria Mycobacterium tuberculosis (Bahuguna, A. & Rawat, D. S. Med Res Rev 40, 263-292, (2020)). Additional research is ongoing into Clofazimine treatment as an anti-cancer agent (Mulkearns-Hubert, E. E. et al. Cell Rep 27, 1062-1072.e1065, (2019)). Its canonical mode of action for these indications is to preferentially bind guanine-rich areas of Mycobacterium DNA and prevent bacterial development. Although it has been shown that there is little interaction of Clofazimine with poly(A) in a previous study (Morrison, N. E. & Marley, G. M. Int J Lepr Other Mycobact Dis 44, 475-481 (1976)), it is a reasonable assumption that Clofazimine could be binding directly to poly(A) and inhibiting poly(A):MSUT2 interaction in the in vitro studies described herein. Further, because it is known that Clofazimine is unable to cross the blood brain barrier, any further translational studies will require generation of brain-penetrant derivatives (Mulkearns-Hubert, E. E. et al. Cell Rep 27, 1062-1072.e1065, (2019)).

Future drug discovery efforts against MSUT2 and other potential targets exacerbating neurodegeneration will require much larger repositories of compounds to find lead candidates as well as discovery of new screening methods and alternative therapeutic strategies. Further, advances are being made in silico or virtual drug screening in regard to algorithms predicting protein:inhibitor conformations as well as in scoring potential therapeutics, highlighting the need for a molecular structure of MSUT2. This so-called virtual docking of ligands to MSUT2 will be an important avenue in increasing throughput for lead candidate discovery. The data described herein demonstrates MSUT2 activity is clearly targetable and a strong candidate for further small molecule screening campaigns.

Methods. RNA. 5′ Fluorescein labeled and unlabeled poly(A) RNA were purchased from IDT (sequences 5′-AAAAAAAAAAAAAAA-3′ (SEQ ID NO: 2) and 5′FAM-AAAAAAAAAAAAAAA-3′ (SEQ ID NO: 3)). Both FAM-labeled and unlabeled RNA were diluted to 100 μM in RNAse/DNAse free Qiagen water and stored at −80° C., away from light.

Recombinant Protein. MSUT2-ZF and PABNP1 cDNA were cloned into the pGEX-6P1 vector (Pharmacia). MSUT2-ZF and PABPN1 encoding plasmids were transformed into BL21 (DE3) bacteria. 10 mL Terrific Broth (TB) starter cultures were grown overnight at 37° C. in a shaking incubator. The following morning, 1 L TB cultures were inoculated and grown at 37° C. with shaking to log phase and induced with 1 mM final concentration IPTG for 3 hours at 37° C. Following induction, DNA and RNA was degraded using benzonase nuclease. Affinity based gravity column purification was performed by binding GST-tagged MSUT2 or PABPN1 to sepharose-glutathione resin by subsequently eluting with 20 mM glutathione. Resulting eluate was buffer exchanged into PBS and stored at −80° C. Protein purity and yield were analyzed via Bradford assay and Coomassie-stained SDS-PAGE.

Chemical Library. The NIH Chemical Collection (NIHCC) was purchased from Evotec and contains a total of 9 96-well plates (700 compounds at 10 mM concentration at 10 uL volumes). For screening, compound was diluted to 250 μM in a separate working drug dilution plate. 2 uL of compound was transferred to 50 uL final assay volume via Integra Viaflo, for a final compound concentration of 10μM.

Fluorescence Polarization Assay. Fluorescence polarization assay was performed in ½ area black plates (Corning 3686). Reaction mixtures were a total volume of 504 and contained final concentrations of 125 nM MSUT2 and 10 nM FAM-RNA in PBS, transferred using an Integra Viaflo with 96/50 uL head. 24 of stock compound was transferred yielding 10 μM final concentration. Plates were then incubated at room temperature, without shaking, for 20 minutes in a BioSpa8 automated incubator and transferred by robotic arm to a Biotek Cytation 5 with pre-configured green polarization filter cube (8040561) at excitation 485/20 emission 528/20 and dichroic mirror at 510 nm and a read height of 10 mm. Fluorescence polarization was calculated by first subtracting background from a buffer-only control well and then using the equation

$$P = \frac{{F}_{∥} - {F}_{⊥}}{{F}_{∥} + {F}_{⊥}}$$

to determine polarization (P).

Alpha Screen Assay. Samples were set up in 96 well Perkin Elmer ½ area white opaque-bottom plates (PE06). A total reaction volume of 504 was used by first adding 20 μL of donor beads (4 μg/mL final concentration), then 10 μL biotinylated RNA (250 nM final concentration) and next 10 μL GST-MSUT2 protein (250 nM final concentration). This mixture was incubated at room temperature for 30 minutes away from light after which 10 μL of acceptor beads (1.25 μg/mL final concentration) was added. The plate was then incubated again at room temperature away from light for 60 minutes and subsequently read on a Perkin Elmer EnSight multimode microplate reader using a standard 96-well Alpha Assay protocol.

Cell culture and assays. Cell viability was assessed. Briefly, HEK-293 cells grown to 70% confluence in a 96 well dish were treated with varying concentrations of compound (final concentration of 2% DMSO) and incubated for 72 hours. Next, Promega Cell Titer Glo assay was used to assess viability per manufacturer instructions (Promega G7570).

Statistical analyses and figures. Graphs were generated using GraphPad Prism 8. IC50 calculations were performed using GraphPad Prism 8 curve fitting using 4-parameter non-linear regression.

1.-17. (canceled)

18. A method of inhibiting expression of a MSUT2 polynucleotide in a subject, the method comprising administering to a subject with Alzheimer's disease, a tauopathy disorder or dementia a therapeutically effective amount of one or more of the mammalian suppressor of tauopathy 2 (MSUT2) inhibitors listed in Table 1.

19. (canceled)

20. A method of reducing phosphorylated and aggregated human tau protein in a subject, the method comprising administering to a subject with Alzheimer's disease, a tauopathy disorder or dementia a therapeutically effective amount one or more of the mammalian suppressor of tauopathy 2 (MSUT2) inhibitors listed in Table 1.

21.-28. (canceled)

29. The method of claim 18, wherein the expression of the MSUT2 polynucleotide is inhibited or suppressed by the one or more of the MSUT2 inhibitors listed in Table 1 is by inhibiting the binding of poly(A) RNA to the MSUT2 polynucleotide.

30. (canceled)

31. The method of claim 18, wherein the one or more of the MSUT2 inhibitors is duloxetine, saquinavir or clofazimine.

32. The method of claim 31, wherein duloxetine, saquinavir or clofazimine has a Ki lower for MSUT2 than for its known target thereby allowing a lower therapeutically effective amount to be administered.

33. The method of claim 29, wherein the one or more MSUT2 inhibitors inhibits MSUT2 binding to poly(A) RNA without altering the poly(A):PABPN1 interaction.

34. The method of claim 18, wherein the one or more MSUT2 inhibitors is administered orally, intramuscularly, intraperitonealy, intravenously, subcutaneously, intrathecally, intranasally, or by direct injection.

35. (canceled)

36. The method of claim 18, further comprising administering a cholinesterase inhibitor to the subject.

37. The method of claim 36, wherein the cholinesterase inhibitor is galantamine, rivastigmine or donepezil.

38.-42. (canceled)

43. The method of claim 20, wherein the tauopathy disorder is Frontotemporal Lobar Degeneration Frontotemporal Dementia (FTLD), primary progressive aphasia, atypical dopaminergic-resistant Parkinsonian syndromes with prominent extra-pyramidal symptoms or corticobasal syndrome.

44. The method of claim 18, wherein the cell is a brain cell.

45.-74. (canceled)

75. A method for identifying a candidate composition capable of inhibiting a RNA binding protein (RBP) binding to poly(A) RNA, the method comprising:
a) contacting a fluorescent probe molecule bound to the poly(A)RNA molecule (FAM-RNA), the RBP, and the candidate composition in a sample under conditions in which the RBP is capable of binding to the FAM-RNA molecule and forming a macromolecular complex, wherein the macromolecular complex comprises FAM-RNA:RBP;
b) exciting fluorescence in the sample with linearly polarized light from a pulsed excitation source;
c) detecting a fluorescent emission from the excited sample; and
d) measuring anisotropy of the emitted fluorescence,
wherein the reduction of emitted polarized fluorescence identifies a candidate composition capable of inhibiting a RNA binding protein (RBP) binding to poly(A) RNA.

76. The method of claim 75, wherein the RBP is a recombinant MSUT2 zinc finger protein.

77. The method of claim 75, wherein the RBP is PABPN1.

78. The method of claim 75, further comprising selecting the candidate compound which inhibits the formation of the macromolecular complex.

79. The method of claim 78, wherein the candidate compound which inhibits the formation of the macromolecular complex inhibits RBP binding to FAM-RNA, wherein the FAM-RNA emits a low level of polarized light.

80. The method of claim 75, wherein the macromolecular complex emits a high level of polarized light.

81. The method of claim 75, wherein the anisotropy of the emitted fluorescence is by determining the intensities of fluorescent emission of the FAM-RNA and/or the macromolecular complex.

82. The method of claim 20, wherein the one or more of the MSUT2 inhibitors is duloxetine, saquinavir or clofazimine, and wherein duloxetine, saquinavir or clofazimine inhibits MSUT2 binding to poly(A) RNA without altering the poly(A):PABPN1 interaction.

83. The method of claim 82, wherein duloxetine, saquinavir or clofazimine has a Ki lower for MSUT2 than for its known target thereby allowing a lower therapeutically effective amount to be administered.


SEQUENCE LISTING (3 sequences)

SEQ ID NO: 1 - DNA, 15 bp, Artificial Sequence
  Synthetic construct
  misc_feature (1)..(1): Biotin at 5′ end
aaaaaaaaaaaaaaa

SEQ ID NO: 2 - DNA, 15 bp, Artificial Sequence
  Synthetic construct
aaaaaaaaaaaaaaa

SEQ ID NO: 3 - DNA, 15 bp, Artificial Sequence
  Synthetic construct
  misc_feature (1)..(1): FAM at 5′ end
aaaaaaaaaaaaaaa